(19) United States
(12) Patent Application Publication
(10) Pub. No.: US 2009/0022694 A1
(43) Pub. Date: Jan. 22, 2009

(54) SIRT1 INHIBITION

(76) Inventor: Peter Distefano, Southboro, MA (US)

Correspondence Address: LOWRIE, LANDO & ANASTASI, LLP;E2023, One Main Street, Suite 1100, Cambridge, MA, 02142, US

(21) Appl. No.: 12/090,167

(22) PCT Filed: Oct. 17, 2006
(86) PCT No.: PCT/US06/40426

§ 371 Date: Sep. 26, 2008

Related U.S. Application Data
(60) Provisional application No. 60/727,651, filed on Oct. 18, 2005.

(51) Int. Cl. A61K 35/12; C12N 5/00; A61P 3/10; A61P 3/00; A61P 25/00; A61P 9/00; A61P 7/02

(52) U.S. Cl. 424/93.7; 435/375

(57) ABSTRACT

A method of culturing cells in the presence of a SIRT1 inhibitor is described.

### CROSS REFERENCE TO RELATED APPLICATIONS

This application claims priority to U.S. Application Ser. No. 60/727,651, filed on Oct. 18, 2005, the contents of which are hereby incorporated by reference in their entirety.

### SUMMARY

A SIRT1 inhibitor can be used to enhance the properties of cells, particularly cultured cells and cells for transplantation. In one aspect, this disclosure features a method of modulating cells, e.g., progenitor cells or other cells described herein. The method includes inhibiting SIRT1 activity in cells, e.g., in vitro. The method can include culturing cells, e.g., in tissue culture. The cells are generally mammalian cells, e.g., mammalian cells that express SIRT1, e.g., human cells. The method can further include maintaining the cells in vitro in the presence of an effective amount of a SIRT1 inhibitor, e.g., a small molecule inhibitor of SIRT1 (e.g., an inhibitor described herein), particularly an inhibitor of a SIRT1 enzymatic activity, e.g., deacetylase activity.

In one embodiment, the SIRT1 inhibitor has an IC₅₀ of 10 μM, 1 μM, 100 nM, 50 nM, 10 nM or less. The maintaining can include combining a culture that includes the cells with a substantially pure preparation of the SIRT1 inhibitor.

The cells can be obtained directly from a subject, can be cells of a primary cell line, or an immortalized cell line. In one embodiment, the cells are not terminally differentiated. In one embodiment, the cells are not transformed (with respect oncogenesis).

The cells can be in the form of isolated cells (e.g., in a sheet or individually in solution or on a substrate), tissue, or an organ, or parts thereof.

The SIRT1 inhibitor can be provided in an amount effective to prolong lifespan of the cells or to increase the replicative capacity of the cells. The term “prolonged lifespan” refers to an increase in the time until a cell terminally differentiates or stops proliferating. The term “replicative capacity” refers to the number of times a cell can divide, e.g., the number of cell divisions until the cell terminally differentiates, stops replicating, senesces, or dies. These parameters can be evaluated by assaying a culture that contains the inhibitor and a control culture to determine the time until terminal differentiation or the end of proliferations, and so forth.

In certain cases, the SIRT1 inhibitor can decrease oxidative damage (e.g., and thereby increase cell survival) associated with a disease (e.g., liver disease) or procedure (e.g., a surgical procedure). For example, the cells can be cells that respond to a SIRT1 inhibitor by having increased resistance to oxidative stress.

An effective amount can be determined by evaluating a range of concentrations of the SIRT1 inhibitor, e.g., to identify one or more concentrations that produce a statistically significant effect. An effective amount of a SIRT1 inhibitor, for example, a SIRT1 inhibitor described herein may range, for example, from a concentration about 0.01-10 times the IC₅₀ of the particular compound, e.g., from 0.1 to 2 times the IC₅₀ when used in culture. Effective doses will also vary depending on the specific culture conditions and cell type, as well as the possibility of co-usage with other agents.

In one embodiment, the cells are transplantable, e.g., they can be administered to a subject, e.g., a subject that was the source of the cells, or a different subject, e.g., a immunosuppressed subject, or a normal subject. For example, the cells can be a form suitable for transplantation, e.g., organ transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

The cells can be, for example, administered to a subject who has experienced or is at risk of experiencing senescence (e.g., abnormal senescence), diabetes (e.g., type I or II), metabolic syndrome, skeletal muscle disease (e.g., Duchene muscular dystrophy, Becker's dystrophy, myotonic dystrophy), ALS under neurodegenerative disease, spinal cord trauma, heart disease, stroke, macular degeneration, a chronically degenerative disease (such as cardiac muscle disease, neurodegenerative disease (e.g., Parkinson's disease, Alzheimer's disease, Huntington's Disease), bone disease (e.g., osteoporosis), a blood disease (e.g., a leukemia) and liver disease (e.g., due to alcohol abuse or hepatitis)), or other condition characterized by unwanted cell loss, or a subject who has undergone chemotherapy or radiation treatment, a subject that has suffered a wound, a burn, an ulcer (e.g., ulcer in a diabetic, e.g., diabetic foot ulcer), a surgical wound, a sore, and abrasions.

In one embodiment, the method further includes contacting a test agent to the cells cultured with the SIRT1 inhibitor. The contacting can be used to characterize the test agent, e.g., a drug or drug candidate, e.g., to evaluate toxicity, a biochemical property, and responsiveness of the cell.

In another aspect, the disclosure features a cell culture medium that includes a buffered medium, growth factors, and a SIRT1 inhibitor, e.g., an inhibitor having an IC₅₀ for SIRT1 enzymatic activity of less than 10 μM, e.g., an inhibitor described herein. The disclosure also features a cell culture that includes mammalian cells (e.g., stem cells, sperm, or eggs) and a SIRT1 inhibitor, and optionally a medium that comprises nutrients and growth factors. In one embodiment, the medium further includes a cryoprotectant, adjuvant, anti-oxidant, and so forth.

In one aspect, a method of culturing cells is described. The method includes inhibiting SIRT1 activity in cells in vitro.

In some embodiments, the method includes maintaining the cells in vitro in the presence of an effective amount of a SIRT1 inhibitor (e.g., a small molecule).

In some embodiments, the method includes combining the cells with a substantially pure preparation of the SIRT1 inhibitor.

In some embodiments, the SIRT1 inhibitor has an IC₅₀ of 10 μM or less.

In some embodiments of the disclosure, the method the cells are mammalian (e.g., human). In some embodiments, the cells are not terminally differentiated. In other embodiments, prior to culturing, the cells are senescent or terminally differentiated. In a preferred embodiment, prior to culturing, the cells are senescent. In another preferred embodiment, prior to culturing, the cells are terminally differentiated. In some embodiments, the cells are not transformed.

In some embodiments, the SIRT1 inhibitor prolongs lifespan of the cells.

In other embodiments, wherein replicative capacity of the cells is increased.

In some embodiments, the cells are transplantable. The cells may be genetically engineered.

In some embodiments, the method also includes administering the cells (e.g., genetically engineered cells) to a subject in need thereof.

In some embodiments, the cells are bone marrow cells, cardiac muscle cells, dopamine-producing cells, osteoblasts, osteocytes, hepatocytes, stromal cells, fetal brain cells, pancreatic B cells, or myoblasts. In preferred embodiments, the cells are cardiac muscle cells, dopamine-producing cells, osteoblasts, osteocytes, hepatocytes, fetal brain cells, pancreatic B cells, or myoblasts. In a more preferred embodiment, the cells are cardiac muscle cells. In a more preferred embodiment, the cells are dopamine-producing cells. In a more preferred embodiment, the cells are osteoblasts. In a more preferred embodiment, the cells are osteocytes. In a more preferred embodiment, the cells are hepatocytes. In a more preferred embodiment, the cells are fetal brain cells. In a more preferred embodiment, the cells are pancreatic B cells. In a more preferred embodiment, the cells are myoblasts.

In other embodiments, the cells are stem cells. In some embodiments, the stem cell is committed to a mesenchymal, hematopoietic, adipogenic, hepatogenic, neurogenic, gliogenic, chondrogenic, vasogenic, myogenic, chondrogenic, or osteogenic lineage. In preferred embodiments, the stem cell is committed to an adipogenic, hepatogenic, neurogenic, gliogenic, vasogenic, myogenic, or osteogenic lineage. In a more preferred embodiment, the stem cell is committed to an adipogenic lineage. In a more preferred embodiment, the stem cell is committed to a hepatogenic lineage. In a more preferred embodiment, the stem cell is committed to a neurogenic lineage. In a more preferred embodiment, the stem cell is committed to a gliogenic lineage. In a more preferred embodiment, the stem cell is committed to a vasogenic lineage. In a more preferred embodiment, the stem cell is committed to a myogenic lineage. In a more preferred embodiment, the stem cell is committed to an osteogenic lineage.

In some embodiments, the cells are administered to a subject who has experienced or is at risk of experiencing abnormal senescence, diabetes (e.g., type I or II), metabolic syndrome, skeletal muscle disease (e.g., Duchene muscular dystrophy, Becker's dystrophy, or myotonic dystrophy), ALS under neurodegenerative disease, spinal cord trauma, heart disease, stroke, macular degeneration, a chronically degenerative disease (such as cardiac muscle disease, neurodegenerative disease (e.g., Parkinson's disease, Alzheimer's disease, or Huntington's Disease), bone disease (e.g., osteoporosis), a blood disease (e.g., a leukemia) or liver disease (e.g., due to alcohol abuse or hepatitis)), or other condition characterized by unwanted cell loss, or a subject who has undergone chemotherapy or radiation treatment, a subject that has suffered a wound (e.g., a surgical wound), a burn, an ulcer (e.g., ulcer in a diabetic, e.g., diabetic foot ulcer), a sore, or abrasions. In preferred embodiments, the cells are administered to a subject who has experienced or is at risk of experiencing abnormal senescence, diabetes (e.g., type I or II), metabolic syndrome, skeletal muscle disease (e.g., Duchene muscular dystrophy, Becker's dystrophy, or myotonic dystrophy), ALS under neurodegenerative disease, spinal cord trauma, heart disease, stroke, macular degeneration, a chronically degenerative disease (such as cardiac muscle disease, neurodegenerative disease (e.g., Parkinson's disease, Alzheimer's disease, or Huntington's Disease), or liver disease (e.g., due to alcohol abuse or hepatitis)), or other condition characterized by unwanted cell loss, or a subject that has suffered an ulcer (e.g., ulcer in a diabetic, e.g., diabetic foot ulcer), a sore, or abrasions.

In a more preferred embodiment, the cells are administered to a subject who has experienced or is at risk of experiencing abnormal senescence. In a more preferred embodiment, the cells are administered to a subject who has experienced or is at risk of experiencing diabetes (e.g., type I or II). In a more preferred embodiment, the cells are administered to a subject who has experienced or is at risk of experiencing metabolic syndrome. In a more preferred embodiment, the cells are administered to a subject who has experienced or is at risk of experiencing skeletal muscle disease (e.g., Duchene muscular dystrophy, Becker's dystrophy, or myotonic dystrophy). In a more preferred embodiment, the cells are administered to a subject who has experienced or is at risk of experiencing ALS under neurodegenerative disease. In a more preferred embodiment, the cells are administered to a subject who has experienced or is at risk of experiencing spinal cord trauma. In a more preferred embodiment, the cells are administered to a subject who has experienced or is at risk of experiencing heart disease. In a more preferred embodiment, the cells are administered to a subject who has experienced or is at risk of experiencing stroke. In a more preferred embodiment, the cells are administered to a subject who has experienced or is at risk of experiencing macular degeneration. In a more preferred embodiment, the cells are administered to a subject who has experienced or is at risk of a chronically degenerative disease (such as cardiac muscle disease, neurodegenerative disease (e.g., Parkinson's disease, Alzheimer's disease, or Huntington's Disease). In a more preferred embodiment, the cells are administered to a subject who has experienced or is at risk of experiencing liver disease (e.g., due to alcohol abuse or hepatitis). In a more preferred embodiment, the cells are administered to a subject who has experienced or is at risk of experiencing a condition characterized by unwanted cell loss. In a more preferred embodiment, the cells are administered to a subject who has suffered or is at risk of suffering with an ulcer (e.g., ulcer in a diabetic, e.g., diabetic foot ulcer). In a more preferred embodiment, the cells are administered to a subject who has experienced or is at risk of experiencing a sore. In a more preferred embodiment, the cells are administered to a subject who has experienced or is at risk of experiencing an abrasion.

In some embodiments, the method also includes evaluating one or more test compounds by contacting the test compound to the cells.

In some aspects of the methods described herein, the SIRT1 inhibitor comprises a compound having formula (I):

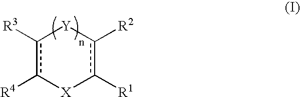

wherein,

R¹ and R², together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which may be optionally substituted with 1-5 R⁵; or R¹ is H, S-alkyl, or S-aryl, and R² is amidoalkyl wherein the nitrogen is substituted with alkyl, aryl, or arylalkyl, each of which is optionally further substituted with alkyl, halo, hydroxy, or alkoxy;

R³ and R⁴, together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which are optionally substituted with 1-5 R⁶;

each of R⁵ and R⁶ is, independently, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, SO₃H, sulfate, S(O)NH₂, S(O)₂NH₂, phosphate, C₁-C₄ alkylenedioxy, oxo, acyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl; alkoxyaminocarbonyl; or one of R⁵ or R⁶ and R⁷ form a cyclic moiety containing 4-6 carbons, 1-3 nitrogens, 0-2 oxygens and 0-2 sulfurs, which are optionally substituted with oxo or C₁-C₆ alkyl;

X is NR⁷, O, or S; Y is NR^{7′}, O or S;

———— represent optional double bonds;

each of R⁷ and R^{7′} is, independently, hydrogen, C₁-C₆ alkyl, C₇-C₁₂ arylalkyl, C₇-C₁₂ heteroarylalkyl; or R⁷ and one of R⁵ or R⁶ form a cyclic moiety containing 4-6 carbons, 1-3 nitrogens, 0-2 oxygens and 0-2 sulfurs, which are optionally substituted with oxo or C₁-C₆ alkyl; and

n is 0 or 1.

In some embodiments, R¹ and R², together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which may be optionally substituted with 1-5 R⁵.

In other embodiments, R¹ and R², together with the carbons to which they are attached, form C₅-C₁₀ cycloalkenyl. In some embodiments, R¹ and R² are substituted with R⁵.

In some embodiments, R³ and R⁴, together with the carbons to which they are attached, form C₆-C₁₀ aryl. In some embodiments, R³ and R⁴ are substituted with R⁶. In some embodiments, R⁶ is halo or C₁-C₆ alkyl.

In some embodiments, n is 0.

In other embodiments, X is NR⁷.

In some preferred embodiments, n is 0 and X is NR⁷.

In some embodiments, the compound of formula (I) compound has the formula (X) below:

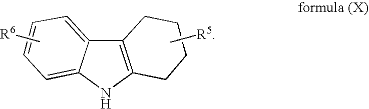

In some embodiments, R⁶ is halo or C₁-C₆ alkyl.

In some embodiments, R⁵ is aminocarbonyl.

In some embodiments, the compound of formula (X) has the formula (XI) below:

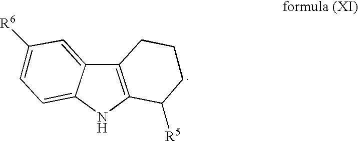

In some embodiments, R⁶ is halo or alkyl.

In some embodiments, R⁵ is aminocarbonyl.

In some preferred embodiments, R⁶ is halo or alkyl and wherein R⁵ is aminocarbonyl.

In some embodiments, the compound of formula (X) is 6-Chloro-2,3,4,9-tetrahydro-1H-carbazole-1-carboxylic acid amide. In some embodiments, the compound contains greater than a 60% (e.g., greater than a 65%; 70%, 75%, 80%, or 85%) enantiomeric excess of the enantiomer having an optical rotation of −14.1 (c=0.33 DCM). In preferred embodiments, the compound contains greater than a 90% (e.g., greater than a 91%, 92%, 93%, 94%, 95%, 96%, 97%, 98%, or 99%) enantiomeric excess of the enantiomer having an optical rotation of −14.1 (c=0.33 DCM).

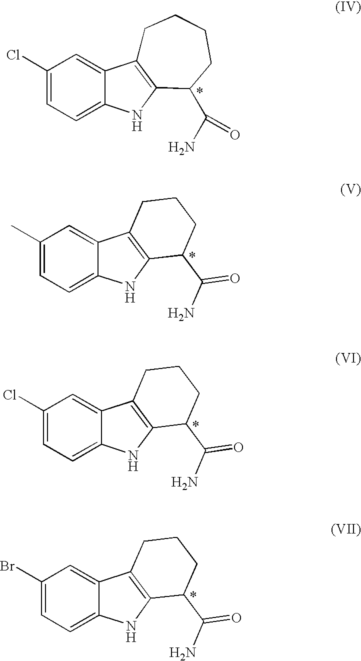

In one instance, the compound can be a compound of formula (VI) having a high enantiomeric excess of a single isomer, wherein the optical rotation of the predominant isomer is negative, for example, −14.1 (c=0.33, DCM) or, for example, [α]_{D}²⁵-41.2° (c 0.96, CH₃OH). In a second instance, the compound can be a compound of formula (IV) having a high enantiomeric excess of a single isomer, wherein the optical rotation of the predominant isomer is negative. In some instances, a compound of formula (IV), (V), or (VII) is administered having a high enantiomeric excess of a single isomer, where the predominant isomer has the same absolute configuration as the negative isomer of the compound of formula (VI) as corresponds to the asterisk carbon shown above.

In some aspects of the methods described herein, the SIRT1 inhibitor includes a compound having formula (XXII):

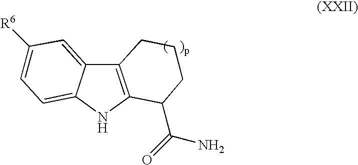

wherein R⁶ is halo or C₁-C₆ alkyl, and

p is 0, 1, or 2; and

wherein, the compound is enriched for the isomer having the same relative stereochemistry as the stereoisomer of 6-Chloro-2,3,4,9-tetrahydro-1H-carbazole-1-carboxylic acid amid having an optical rotation of −14.1 (c=0.33 DCM) (e.g., the compound has an enantiomeric excess of at least about 55%, 60%, 65%, 70%, 75%, 80%, 85%, 90%, 95%, 98%, or 99%).

In some embodiments, R⁶ is chloro or methyl.

In some embodiments, p is 1.

In some aspects of the methods described herein, the SIRT1 inhibitor includes a compound that is enriched for a stereoisomer of 6-Chloro-2,3,4,9-tetrahydro-1H-carbazole-1-carboxylic acid amid having an optical rotation of −14.1 (c=0.33 DCM). In some aspects of the methods described herein, the SIRT1 inhibitor includes an enriched form of a stereoisomer of 6-Chloro-2,3,4,9-tetrahydro-1H-carbazole-1-carboxylic acid amid having an optical rotation of −14.1 (c=0.33 DCM).

In some aspects of the methods described herein, the SIRT1 inhibitor includes a compound having formula (XXIII):

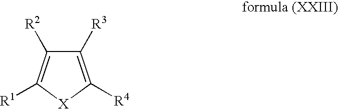

wherein

R¹ is H, halo, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl; or when taken together with R² and the carbon to which it is attached, forms C₅-C₁₀ cycloalkenyl, C₅-C₁₀ heterocycloalkenyl, C₆-C₁₀ aryl, or C₆-C₁₀ heteroaryl; each of which can be optionally substituted with 1-5 R⁵;

R² is H, halo, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl; or when taken together with R² and the carbon to which it is attached, forms C₅-C₁₀ cycloalkenyl, C₅-C₁₀ heterocycloalkenyl, C₆-C₁₀ aryl, or C₆-C₁₀ heteroaryl; each of which can be optionally substituted with 1-5 R⁶;

each of R³ and R⁴ is, independently, H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃R⁹, sulfate, S(O)N(R⁹)₂, S(O)₂N(R⁹)₂, phosphate, C₁-C₄ alkylenedioxy, acyl, amido, aminocarbonyl, C₁-C₆ alkyl aminocarbonyl, C₁-C₆ dialkyl aminocarbonyl, aminocarbonylalkyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl; each of which is independently substituted with one or more R⁷;

each or R⁵ and R⁶ is, independently, halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, oxo, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃R⁹, sulfate, S(O)N(R⁹)₂, S(O)₂N(R⁹)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl;

each R⁷ is independently C₁-C₁₀ alkyl, C₁-C₆ haloalkyl, aminocarbonyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₇-C₁₂ heterocyclylalkyl, C₇-C₁₂ cyloalkylalkyl, C₇-C₁₂ heterocycloalkenylalkyl, or C₇-C₁₂ cycloalkenylalkyl; each of which is optionally substituted with 1-4 R¹⁰;

X is NR⁸, O, or S;

R⁸ is H, C₁-C₆ alkyl, C₆-C₁₀ aryl, C₅-C₁₀ heteroaryl, C₇-C₁₂ arylalkyl, C₇-C₁₂ heteroarylalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₇-C₁₂ heterocyclylalkyl, C₇-C₁₂ cyloalkylalkyl, C₇-C₁₂ heterocycloalkenylalkyl, or C₇-C₁₂ cycloalkenylalkyl;

R⁹ is H or C₁-C₆ alkyl; and

each R¹⁰ is independently halo, hydroxy, alkoxy, alkyl, alkenyl, alkynl, nitro, amino, cyano, amido, or aminocarbonyl.

In some embodiments, R¹ and R², taken together, with the carbons to which they are attached, form C₅-C₁₀ cycloalkenyl, C₅-C₁₀ heterocycloalkenyl, C₆-C₁₀ aryl, or C₆-C₁₀ heteroaryl. In some preferred embodiments, R¹ and R², taken together, with the carbons to which they are attached, form C₅-C₁₀ cycloalkenyl. In some preferred embodiments, R¹ and R², taken together, with the carbons to which they are attached, form C₅-C₁₀ cycloalkenyl, optionally substituted with 1 or 2 C₁-C₆ alkyl. In some preferred embodiments, R¹ and R², taken together form a C₅-C₇ cycloalkenyl ring substituted with C₁-C₆ alkyl.

In some embodiments, R¹ is C₆-C₁₀ aryl, C₅-C₁₀ heteroaryl, C₇-C₁₂ aralkyl, C₇-C₁₂ heteroaralkyl, C₃-C₈ heterocyclyl, C₅-C₁₀ cycloalkenyl, or C₅-C₁₀ heterocycloalkenyl. In some preferred embodiments, R¹ is C₆-C₁₀ aryl.

In some embodiments, R² is H, halo, C₁-C₁₀ alkyl, or C₁-C₆ haloalkyl.

In some embodiments, R³ is carboxy, cyano, aminocarbonyl, C₁-C₆ alkyl aminocarbonyl, C₁-C₆ dialkyl aminocarbonyl, C₁-C₁₀ alkoxycarbonyl, C₁-C₁₀ alkylthioylcarbonyl, hydrazinocarbonyl, C₁-C₆ alkylhydrazinocarbonyl, C₁-C₆ dialkyl hydrazinocarbonyl, or hydroxyaminocarbonyl. In some preferred embodiments, R³ is aminocarbonyl, C₁-C₆ alkyl aminocarbonyl, C₁-C₆ dialkyl aminocarbonyl, hydrazinocarbonyl, C₁-C₆ alkyl hydrazinocarbonyl, C₁-C₆ dialkyl hydrazinocarbonyl, or hydroxyaminocarbonyl. In some preferred embodiments, R³ is aminocarbonyl, C₁-C₆ alkyl aminocarbonyl, or C₁-C₆ dialkyl aminocarbonyl.

In some embodiments, R³ is H, thioalkoxy or thioaryloxy.

In some embodiments, R⁴ is nitro, amino, C₁-C₆ alkyl amino, C₁-C₆ dialkyl amino, or amido. In some preferred embodiments, R⁴ is amino or amido.

In some embodiments, R⁴ is aminocarbonylalkyl. In some preferred embodiments, amino of the aminocarbonylalkyl is substituted with aryl, arylalkyl, alkyl, etc. In some preferred embodiments, each substituent can independently be further substituted with halo, hydroxy, or alkoxy.

In some embodiments, R³ is aminocarbonyl, C₁-C₆ alkyl aminocarbonyl, or C₁-C₆ dialkyl aminocarbonyl; and R⁴ is amino, C₁-C₆ alkyl amino C₁-C₆ dialkyl amino or amido.

In some embodiments, X is S.

In some embodiments, X is NR⁸. In some preferred embodiments, R⁸ is H, C₁-C₆ alkyl or C₇-C₁₀ arylalkyl.

In some embodiments, R¹ is C₆-C₁₀ aryl, C₅-C₁₀ heteroaryl, C₇-C₁₂ aralkyl, C₇-C₁₂ heteroaralkyl, C₃-C₈ heterocyclyl, C₅-C₁₀ cycloalkenyl, or C₅-C₁₀ heterocycloalkenyl; or when taken together with R² and the carbon to which it is attached, forms C₅-C₁₀ cycloalkenyl;

R² is H, halo, C₁-C₁₀ alkyl, C₁-C₆ haloalkyl; or when taken together with R¹ and the carbon to which it is attached, forms C₅-C₁₀ cycloalkenyl;

R³ is aminocarbonyl, C₁-C₆ alkyl aminocarbonyl, C₁-C₆ dialkyl aminocarbonyl, hydrazinocarbonyl, C₁-C₆ alkyl hydrazinocarbonyl, C₁-C₆ dialkyl hydrazinocarbonyl, or hydroxyaminocarbonyl;

R⁴ is amino, C₁-C₆ alkyl amino, C₁-C₆ dialkyl amino, or amido; and

X is S.

In some embodiments, R¹ and R², taken together with the carbons to which they are attached, form C₅-C₁₀ cycloalkenyl;

R³ is aminocarbonyl, C₁-C₆ alkyl aminocarbonyl, or C₁-C₆ dialkyl aminocarbonyl;

R⁴ is amino, C₁-C₆ alkyl amino, C₁-C₆ dialkyl amino, or amido; and

X is S.

In some aspects of the methods described herein, the SIRT1 inhibitor includes a compound having formula (II):

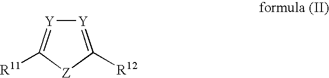

wherein

R¹¹ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃(R¹³), sulfate, S(O)N(R¹³)₂, S(O)₂N(R¹³)₂, phosphate, C₁-C₄ alkylenedioxy, acyl, amido, aminocarbonyl, aminocarbonylalkyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl; wherein each is optionally substituted with R¹⁴;

R¹² is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃(R³), sulfate, S(O)N(R³)₂, S(O)₂N(R³)₂, phosphate, C₁-C₄ alkylenedioxy, acyl, amido, aminocarbonyl, aminocarbonylalkyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, or hydroxyaminocarbonyl or alkoxyaminocarbonyl; wherein each is optionally substituted with R¹⁵;

R¹³ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl;

R¹⁴ is hydroxy, carboxy, carboxylate, cyano, nitro, amino, C₁-C₆ alkyl amino, C₁-C₆ dialkyl amino, oxo, mercapto, thioalkoxy, thioaryloxy, thioheteroaryloxy, SO₃H, sulfate, S(O)NH₂, S(O)₂NH₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl;

R¹⁵ is halo, hydroxy, C₁-C₁₀ alkyl, C₁-C₆ haloalkyl, C₁-C₁₀ alkoxy, C₁-C₆ haloalkoxy, C₆-C₁₀ aryloxy, C₅-C₁₀ heteroaryloxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ arylalkoxy, or C₅-C₁₀ heteroarylalkoxy;

Z is NR¹⁶, O, or S;

each Y is independently N or CR¹⁸;

R¹⁶ is H, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₅-C₁₀ cycloalkenyl, C₅-C₁₀ heterocycloalkenyl, C₂-C₁₂ alkenyl, C₂-C₁₂ alkynyl; or one of R¹¹ or R¹² and R¹⁶ form a cyclic moiety containing 4-6 carbons, 1-3 nitrogens, 0-2 oxygens and 0-2 sulfurs; wherein each is optionally substituted with R¹⁷;

R¹⁷ is halo, hydroxy, C₁-C₆ alkyl, C₁-C₆ haloalkyl, C₁-C₆ alkoxy, C₁-C₆ haloalkoxy, C₂-C₈ alkenyl, C₂-C₈ alkynyl, oxo, mercapto, thioalkoxy, SO₃H, sulfate, S(O)NH₂, S(O)₂NH₂, phosphate, acyl, amido, aminocarbonyl, C₁-C₆ alkyl aminocarbonyl, C₁-C₆ dialkyl aminocarbonyl, C₁-C₆ alkoxycarbonyl, C₁-C₆ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl; and

R¹⁸ is H, halo, or C₁-C₆ alkyl.

In some embodiments, Z is NR¹⁶. In some preferred embodiments, Z is NR¹⁶, and R¹⁶ is C₁-C₁₀ alkyl, cycloalkenyl, C₅-C₁₀ heterocycloalkenyl, C₆-C₁₀ aryl, C₅-C₁₀ heteroaryl, C₇-C₁₂ aralkyl, or C₇-C₁₂ heteroaralkyl. In some preferred embodiments, R¹⁶ is C₁-C₁₀ alkyl, C₆-C₁₀ aryl, C₅-C₁₀ heteroaryl, C₇-C₁₂ aralkyl, or C₇-C₁₂ heteroaralkyl, substituted with one or more halo, alkyl, or alkoxy.

In some embodiments, R¹¹ is mercapto, thioalkoxy, thioaryloxy, thioheteroaryloxy, SO₃(R¹³), sulfate, S(O)N(R¹³)₂, S(O)₂N(R¹³)₂. In some preferred embodiments, R¹¹ is thioalkoxy, thioaryloxy, thioheteroaryloxy. In some preferred embodiments, R¹¹ is thioalkoxy, thioaryloxy, thioheteroaryloxy; substituted with one or more acyl, amido aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl. In some preferred embodiments, R¹¹ is thioalkoxy substituted with one or more amido, aminocarbonyl, C₁-C₆ alkyl aminocarbonyl, or C₁-C₆ dialkyl aminocarbonyl. In more preferred embodiments, R¹¹ is thioalkoxy substituted with aminocarbonyl.

In some embodiments, R¹² is C₁-C₁₀ alkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl. In some preferred embodiments, R¹² is C₁-C₁₀ alkyl, C₆-C₁₀ aryl, C₅-C₁₀ heteroaryl, C₇-C₁₂ aralkyl, or C₇-C₁₂ heteroaralkyl. In some preferred embodiments, R¹² is C₁-C₁₀ alkyl substituted with one or more halo, hydroxy, C₁-C₁₀ alkyl, C₁-C₆ haloalkyl, C₁-C₁₀ alkoxy, C₆-C₁₀ aryloxy, or C₅-C₁₀ heteroaryloxy. In more preferred embodiments, R¹² is C₁-C₁₀ alkyl substituted with aryloxy.

In some embodiments, each Y is N.

In some embodiments, R¹¹ is thioalkoxy, thioaryloxy, thioheteroaryloxy; substituted with one or more acyl, amido aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl; R¹² is C₁-C₁₀ alkyl substituted with one or more halo, hydroxy, C₁-C₁₀ alkyl, C₁-C₆ haloalkyl, C₁-C₁₀ alkoxy, C₆-C₁₀ aryloxy, or C₅-C₁₀ heteroaryloxy; Z is NR¹⁶; each Y is N; and R¹⁶ is C₁-C₁₀ alkyl, C₆-C₁₀ aryl, C₅-C₁₀ heteroaryl, C₇-C₁₂ aralkyl, or C₇-C₁₂ heteroaralkyl, substituted with one or more halo, alkyl, or alkoxy.

In some aspects of the methods described herein, the SIRT1 inhibitor includes a compound having formula (III):

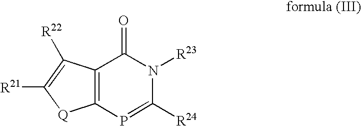

wherein

R²¹ is halo, C₁-C₁₀ alkyl, C₁-C₆ haloalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryl, C₅-C₁₀ heteroaryl, C₇-C₁₂ aralkyl, C₇-C₁₂ heteroaralkyl; or when taken together with R²² and the carbon to which it is attached, forms C₅-C₁₀ cycloalkenyl, C₅-C₁₀ heterocycloalkenyl, C₆-C₁₀ aryl, or C₅-C₁₀ heteroaryl; each of which can be optionally substituted with 1-5 R²⁵;

R²² is halo, C₁-C₁₀ alkyl, C₁-C₆ haloalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryl, C₅-C₁₀ heteroaryl, C₇-C₁₂ aralkyl, C₇-C₁₂ heteroaralkyl; or when taken together with R²¹ and the carbon to which it is attached, forms C₅-C₁₀ cycloalkenyl, C₅-C₁₀ heterocycloalkenyl, C₆-C₁₀ aryl, or C₅-C₁₀ heteroaryl; each of which is optionally substituted with 1-5 R²⁶;

R²³ is H, halo, hydroxy, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, amino, C₁-C₆ alkyl amino, C₁-C₆ dialkyl amino, acyl, C₁-C₁₀ alkoxycarbonyl, C₁-C₁₀ thioalkoxycarbonyl;

R²⁴ is, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, amino, C₁-C₆ alkyl amino, C₁-C₆ dialkyl amino, mercapto, thioalkoxy, thioaryloxy, thioheteroaryloxy, acyl, or amidyl; each of which is optionally substituted with R²⁷;

each R²⁵ and R²⁶ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, oxo, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R²⁸)₂, S(O)₂N(R²⁸)₂, phosphate, C₁-C₄ alkylenedioxy, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl;

R²⁷ is halo, hydroxy, carboxy, carboxylate, oxo, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R²⁸)₂, S(O)₂N(R²⁸)₂, phosphate, C₁-C₄ alkylenedioxy, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl;

R²⁸ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl;

Q is S, O, or NR²⁹; R²⁹ is H, C₁-C₆ alkyl, C₇-C₁₂ aralkyl, or C₇-C₁₂ heteroaralkyl;

P is N or CR³⁰; and

R³⁰ is H or C₁-C₆ alkyl.

In some embodiments, R²¹ and R²², together with the carbons to which they are attached, form C₅-C₁₀ cycloalkenyl, C₅-C₁₀ heterocycloalkenyl, C₆-C₁₀ aryl, or C₅-C₁₀ heteroaryl. In some preferred embodiments, R²¹ and R²², together with the carbons to which they are attached, form C₅-C₁₀ cycloalkenyl.

In some embodiments, R²³ is hydroxy, C₁-C₁₀ alkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, C₁-C₆ dialkyl amino, or acyl. In preferred embodiments, R²³ is C₃-C₈ cycloalkyl, C₅-C₈ heterocyclyl, C₅-C₁₀ cycloalkenyl, or C₅-C₁₀ heterocycloalkenyl.

In some embodiments, R²⁴ is halo, hydroxy, C₁-C₁₀ alkyl, C₁-C₆ haloalkyl, C₁-C₁₀ alkoxy, C₁-C₆ haloalkoxy, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, C₁-C₆ alkyl amino, C₁-C₆ dialkyl amino, mercapto, thioalkoxy, thioaryloxy, or thioheteroaryloxy. In some preferred embodiments, R²⁴ is C₁-C₁₀ alkyl, thioalkoxy, thioaryloxy, or thioheteroaryloxy. In preferred embodiments, R²⁴ is C₁-C₁₀ alkyl or thioalkoxy; and R²⁷ is carboxy, carboxylate, cyano, nitro, amino, C₁-C₆ alkyl amino, C₁-C₆ dialkyl amino, SO₃H, sulfate, S(O)N(R²⁸)₂, S(O)₂N(R²⁸)₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl. In more preferred embodiments, R²⁴ is C₁-C₁₀ alkyl or thioalkoxy; substituted with carboxy, carboxylate, amidyl, or aminocarbonyl.

In some embodiments, X is S.

In some embodiments, Y is N.

In some embodiments, R²¹ and R²², together with the carbons to which they are attached, form C₅-C₁₀ cycloalkenyl, C₅-C₁₀ heterocycloalkenyl, C₆-C₁₀ aryl, or C₅-C₁₀ heteroaryl; R²³ is hydroxy, C₁-C₁₀ alkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, C₁-C₆ dialkyl amino, or acyl; R²⁴ is C₁-C₁₀ alkyl, thioalkoxy, thioaryloxy, or thioheteroaryloxy; R²⁷ is carboxy, carboxylate, cyano, nitro, amino, C₁-C₆ alkyl amino, C₁-C₆ dialkyl amino, SO₃H, sulfate, S(O)N(R²⁸)₂, S(O)₂N(R²⁸)₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl; Q is S; and P is N.

In some embodiments, R²¹ and R²², together with the carbons to which they are attached, form C₅-C₁₀ cycloalkenyl, or C₅-C₁₀ heterocycloalkenyl; R²³ is CL-CIO alkyl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, or C₁-C₆ dialkyl amino; R²⁴ is C₁-C₁₀ alkyl, thioalkoxy, thioaryloxy, or thioheteroaryloxy; R²⁷ is carboxy, carboxylate, SO₃H, sulfate, S(O)N(R²⁸)₂, S(O)₂N(R²⁸)₂, phosphate, aminocarbonyl, C₁-C₆ alkyl aminocarbonyl, C₁-C₆ dialkyl aminocarbonyl, or C₁-C₁₀ alkoxycarbonyl; Q is S; and P is N.

In some aspects of the methods described herein, the SIRT1 inhibitor includes a compound having formula (IV):

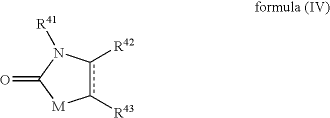

wherein;

R⁴¹ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, amino, C₁-C₆ alkyl amino, C₁-C₆ dialkyl amino, acyl, aminocarbonyl, C₁-C₆ alkyl aminocarbonyl, C₁-C₆ dialkyl aminocarbonyl, C₁-C₁₀ alkoxycarbonyl, or C₁-C₁₀ thioalkoxycarbonyl; each of which is optionally substituted with one or more R⁴⁴;

R⁴² and R⁴³, together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which is optionally substituted with 1-4 R⁴⁵; or

R⁴⁴ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R⁴⁶)₂, S(O)₂N(R⁴⁶)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, or hydroxyaminocarbonyl or alkoxyaminocarbonyl;

R⁴⁵ is halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, oxo, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R⁴⁶)₂, S(O)₂N(R⁴⁶)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl;

R⁴⁶ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl; and

M is NR⁴⁷, S, or O;

R⁴⁷ is H, halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, carboxy, carboxylate, amino, C₁-C₆ alkyl amino, C₁-C₆ dialkyl amino, acyl, aminocarbonyl, C₁-C₆ alkyl aminocarbonyl, C₁-C₆ dialkyl aminocarbonyl, or C₁-C₁₀ alkoxycarbonyl.

In some embodiments, R⁴² and R⁴³, together with the carbons to which they are attached, form C₆-C₁₀ aryl, or C₆-C₁₀ heteroaryl. In some preferred embodiments, R⁴² and R⁴³, together with the carbons to which they are attached, form phenyl. In more preferred embodiments, R⁴² and R⁴³, together with the carbons to which they are attached, form phenyl; and are substituted with halo or C₁-C₁₀ alkyl.

In some embodiments, R¹ is C₁-C₁₀ alkyl; and R⁴⁴ is H, halo, C₆-C₁₀ aryl, C₅-C₁₀ heteroaryl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, acyl, amino, C₁-C₆ alkyl amino, C₁-C₆ dialkyl amino, amido, aminocarbonyl, C₁-C₆ alkyl aminocarbonyl, C₁-C₆ dialkyl aminocarbonyl, carboxy, or C₁-C₁₀ alkoxycarbonyl.

In some embodiments, wherein M is O.

In some embodiments, R⁴¹ is C₁-C₁₀ alkyl; and R⁴⁴ is acyl, amino, C₁-C₆ alkyl amino, C₁-C₆ dialkyl amino, amido, aminocarbonyl, C₁-C₆ alkyl aminocarbonyl, C₁-C₆ dialkyl aminocarbonyl, carboxy, or C₁-C₁₀ alkoxycarbonyl; R⁴² and R⁴³, together with the carbons to which they are attached, form C₆-C₁₀ aryl, or C₆-C₁₀ heteroaryl; and M is O.

In some aspects of the methods described herein, the SIRT1 inhibitor includes a compound having formula V:

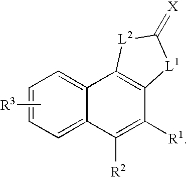

formula (V)

wherein X is a member selected from the group consisting of O and S; L¹ and L² each represent members independently selected from the group consisting of O, S, ethylene and propylene, substituted with 0-2 R groups, wherein exactly one of L¹ and L² represents a member selected from the group consisting of O and S; each instance of R and of L¹ and L² independently represents a member selected from the group consisting of C₁₋₆alkyl, C₂₋₆alkenyl and —CO₂R⁴; R¹ and R² each represent members independently selected from the group consisting of hydrogen, C₁₋₆alkoxy, C₀₋₆ alkoxy-aryl and hydroxyl; R³ is selected from the group consisting of hydrogen, C₁₋₆alkyl, aryl, —OR⁴, —NR⁴R⁴, —CO₂R⁴, —C(O)R⁴, —C(O)NR⁴R⁴, —CN, —NO₂ and halogen; R⁴ independently is selected from the group consisting of hydrogen and C₁₋₆alkyl.

In other aspects of the methods described herein, the SIRT1 inhibitor includes a compound having formula V:

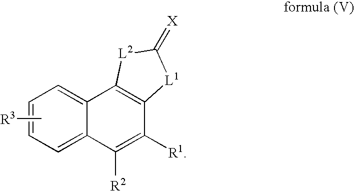

wherein X is a member selected from the group consisting of O and S; L¹ and L² each represent members independently selected from the group consisting of O, S, ethylene and propylene, substituted with 0-2 R groups, wherein exactly one of L¹ and L² represents a member selected from the group consisting of O and S; each instance of R and of L¹ and L² independently represents a member selected from the group consisting of C₁₋₆alkyl, C₂₋₆alkenyl and —CO₂R⁴; R³ is selected from the group consisting of hydrogen, C₁₋₆alkyl, aryl, —OR⁴, —NR⁴R⁴, —CO₂R⁴, —C(O)R⁴, —C(O)NR⁴R⁴, —CN, —NO₂ and halogen; R⁴ independently is selected from the group consisting of hydrogen and C₁₋₆alkyl; R¹ and R² taken together with the carbons to which they are attached form a six-membered lactone ring.

In some embodiments of formula (V), the compound has the following structure:

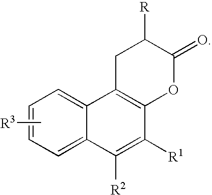

wherein R¹ is a member selected from the group consisting of hydrogen, C₁₋₆alkoxy and C₀₋₆alkoxy-aryl; R² is selected from the group consisting of hydrogen and hydroxy; R³ is selected from the group consisting of hydrogen and —OR⁴; and R⁴ is C₁₋₆ alkyl.

In some embodiments, R¹ is a member selected from the group consisting of C₁₋₆ alkoxy, C₀₋₆ alkoxy-aryl and hydroxy. In some preferred embodiments, R¹ is selected from the group consisting of hydroxy, methoxy and benzyloxy. In other embodiments, the term aryl is selected from the group consisting of phenyl and naphthyl.

In other aspects of the methods described herein, the SIRT1 inhibitor includes a compound having formula VI:

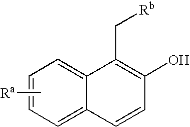

wherein R^{a} is selected from the group consisting of hydrogen, C₁₋₆alkyl, aryl, —OR^{e}, —NR^{e}R^{e}, —CO₂R^{e}, —C(O)R^{e}, —C(O)NR^{e}R^{e}, —CN, —NO₂ and halogen; and R^{b} is selected from the group consisting of:

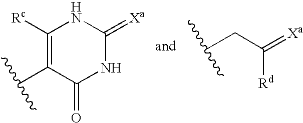

wherein X^{a} can be O, S, or NR^{e}; R^{c} can be hydrogen, C₁₋₆alkyl and aryl optionally substituted with a hydrogen, C₁₋₆alkyl, aryl, -Ore, —NR^{e}R^{e}, —CN, —NO₂ or halogen; R^{d} can be hydrogen, C₁₋₆alkyl, aryl, -Ore, —NR^{e}R^{e}, or halogen, wherein each instance of R^{e} can be independently hydrogen or C₁₋₆ alkyl.

In some embodiments, the SIRT1 inhibitor comprising a compound having formula VI has the following structure

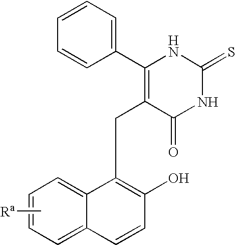

In another aspect, the disclosure features a cell culture medium that includes a buffered medium, growth factors; and a SIRT1 inhibitor having an IC₅₀ for SIRT1 enzymatic activity of less than 10 μM. The SIRT1 inhibitor can be, e.g., a SIRT1 inhibitor described herein.

In another aspect, the disclosure features a cell culture that includes mammalian cells; a medium that contains nutrients, growth factors, and a SIRT1 inhibitor having an IC₅₀ for SIRT1 enzymatic activity of less than 10 μM. The SIRT1 inhibitor can be, e.g., a SIRT1 inhibitor described herein.

Also described here is a packaged product. The packaged product includes a container, a SIRT1 inhibitor in the container, and a legend (e.g., a label or insert) associated with the container and indicating how to use the SIRT1 inhibitor to culture cells, including any of those cells delineated herein.

In another aspect, the disclosure features a method of preparing a donor of cells for transplantation. The method includes administering a SIRT1 inhibitor (e.g., an inhibitor described herein) to the donor, and then obtaining cells from the donor for transplantation. The method can further include transplanting the cells into a recipient. For example, the donor is a mammal, e.g., a pig, a primate, or a human. The donor and the recipient can be the same species, or different species.

In still another aspect, the disclosure features a method of treating a recipient of transplanted cells. The method includes: administering a SIRT1 inhibitor (e.g., an inhibitor described herein) to the recipient, e.g., before, during, or after the recipient receives the transplanted cells. The inhibitor can be provided chronically, e.g., at regular intervals or continuous. For example, the recipient is a mammal, e.g., a pig, a primate, or a human.

In one aspect, the disclosure features a SIRT1 inhibitor (e.g., an inhibitor described herein) for the enhancement of properties of cells, particularly cultured cells and cells for transplantation. In another aspect, the disclosure features use of a SIRT1 inhibitor described herein for the manufacture of a medicament for the enhancement of properties of cells, particularly cultured cells and cells for transplantation. In one embodiment, the medicament is administered to an organ donor prior to transplantation, or to a recipient, e.g., before, during, or after transplantation.

All references cited herein (including articles, patents, patent applications and patent publications, and databases) are incorporated by reference in their entirety.

The details of one or more embodiments of the invention are set forth in the description below. Other features, objects, and advantages of the invention will be apparent from the description and drawings, and from the claims.

### DETAILED DESCRIPTION

The methods presented herein can be used to improve cell characteristics, e.g., to prolong the lifespan of the cells, particularly cells in culture. An exemplary method includes contacting a SIRT1 inhibitor to a cell, e.g., a mammalian cell. Cultured cells that have been treated with a SIRT1 inhibitor can be transplanted into a subject in need of such a transplant, or can be used in vitro, e.g., to maintain or produce cells for subsequent transplantation, to maintain or produce a cell line of interest, to cryopreserve cells, or to characterize compounds (particularly drugs and drug candidates) for a biological property (e.g., toxicity or cell responsiveness). The methods can be used with a wide variety of cells and with a number of SIRT1 inhibitors, for example, as further described below.

### Cells

Virtually any cell type can be cultured by the methods described herein, e.g., with a SIRT1 inhibitor. The cell can be a cell that expresses SIRT1, e.g., a cell that normally expresses SIRT1 or that expresses SIRT1 as a result of a pathological state. The cells can be of any cell type or lineage, from any tissue, from an adult, infant, fetus, or embryo. The cells can be of any mammalian species. The cells can be pluripotent, multipotent, committed to a cell lineage, differentiating, senescent, quiescent, or terminally differentiated. The cells can be transgenic, e.g., a cell into which a gene has been introduced. The cells can be nullizygous, e.g., in which a gene locus has been disrupted. The cells can be proliferating cells. Further, in some cases, the proliferative capacity of cells may be enhanced or restored (for example, the cells were previously quiescent or senescent) by a SIRT1 inhibitor. In one embodiment, the cells are preferably not transformed or derived from a tumor or cancer sample.

The cells can be primary cells obtained from a subject (e.g., a subject (e.g., a mammal, e.g., a human) to whom the cells will be re-introduced, a donor (e.g., a blood donor or biopsy sample)), an established cell line, or cells recently isolated from a subject that have not yet passed through crisis to become an established cell line. Cells can be obtained from a subject who is undergoing surgery, e.g., tissue can be removed during surgery; from a blood or plasma sample, e.g., obtained by venous removal with a syringe or from a wound; from a sample obtained by swabbing the interior of a subject's mouth of other orifice; or from a biopsy, e.g., a punch biopsy.

Cells, particularly mammalian cells, can be selected using a variety of different techniques and settings. Exemplary techniques include flow cytometry sorting (e.g., fluorescence activated cell sorting), antibody-based retention (e.g., on a magnetic bead), and microdissection. Selection techniques include both positive and negative selections or a combination of both techniques. Selections can include one or more repeated steps. A description of positive and negative selection techniques can be found in, for example, U.S. Pat. Nos. 5,925,567, 6,338,942, 6,103,522, 6,117,985, 6,127,135, 6,200,606, 6,342,344, 6,008,040, 5,877,299, 5,814,440, 5,763,266, and 5,677,136.

These cells can then be cultured, e.g., as would cells of a primary cell line. The cells can be treated with a SIRT1 inhibitor, e.g., by adding the SIRT1 inhibitor to the culture medium, e.g., before or after the cells acquire the properties of a primary cell line. In some implementations, primary cell lines are obtained by culturing cells explanted from a subject and treating the cells with a SIRT1 inhibitor prior to passage through the crisis point used to create an established cell line. The cells (e.g., precursor cells (e.g., stem cells or other progenitor cells), sperm, eggs, cultured cells) can be cryopreserved, e.g., stored under liquid nitrogen, in the presence of a SIRT1 inhibitor, for future use (e.g., implantation, transplantation, in vitro culturing or in vitro manipulation, e.g., in vitro fertilization).

Precursor Cells. In one embodiment, the cells are precursor cells, e.g., stem or other progenitor cells. The cells can be obtained, e.g., directly from tissues of an individual, from cell lines, or from less differentiated precursor cells. An exemplary method for obtaining precursor cells from less differentiated cells is described in Gilbert, 1991, Developmental Biology, 3rd Edition, Sinauer Associates, Inc., Sunderland, Mass. The precursor cells can be from any animal, e.g., mammalian, e.g., human, and can be from primary tissue, cell lines, or another source. The precursor cells can be, for example, of ectodermal, mesodermal or endodermal origin.

In one embodiment, the precursor cell is a stem cell. Examples of stem cells include hematopoietic stem cells (HSC; e.g., long term repopulating HSCs), stem cells of epithelial tissues such as the skin and the lining of the gut, embryonic heart muscle cells, liver stem cells, kidney stem cells, and neural stem cells (Stemple and Anderson, Cell 71:973-985, 1992). The stem cells can be expanded in the presence of a SIRT1 inhibitor and under conditions that promote proliferation of the cells. Examples of useful HSCs are described in, for example, U.S. Pat. Nos. 5,763,197, 5,750,397, 5,716,827, 5,194,108, 5,061,620, and 4,714,680.

Exemplary progenitor cells include pluripotent and multipotent stem cells and stromal cells, and include cells, e.g., stem cells that are committed to a particular cell lineage, e.g., mesenchymal, hematopoietic, adipogenic, hepatogenic, neurogenic, gliogenic, chondrogenic, vasogenic, myogenic, chondrogenic, or osteogenic lineage. For example, stromal cells may be used to promote specific differentiation pathways such as those present in the brain, eye, pharyngeal pancreas, lungs, kidneys, liver, heart, intestine, pancreas, bone, cartilage, skeletal muscle, smooth muscle, ear, esophagus, stomach, blood vessels, and aorta-mesonephros (AGM) region (see e.g., US 2005-0153443).

Another type of precursor cell is a primordial germ cell (PGC). PGCs are precursor cells of the germ line in the developing embryo. In mice, primordial germ cells isolated during their migratory phase and cultured on feeders layers (e.g., fetal fibroblast cells (STO cells), Kawase et al., Experimental Medicine 10 (13):1575-1580, 1992) with leukaemia inhibitory factor (LIF) and mouse stem cell factor (MSCF) provide mice PGCs that result in cell lines for long-term culture. In addition, mice PGCs can also be cultured in similar media to which basic fibroblast growth factor (bFGF) has been added, thereby converting PGCs to cells that resemble undifferentiated embryonic stem cells (ESCs) (Matsui et al., Cell 70:5:841-847, 1992; Resnick et al., Nature 359 (6395):550-551, 1992).

An example of PGCs include porcine stem cells. See, e.g., U.S. Pat. No. 6,703,209. Porcine PGCs can be extracted from swine fetuses during about 17 to 39 days post fertilization of the embryo. Preferably, 27 day old fetuses are utilized and PGC suspensions are prepared by trypsin (or EDTA) treatment of genital ridges from the porcine embryos (crossbred) and then seeded on feeder cells (for example in 4-well dishes). The feeder cells are mitotically inactivated and may be STO cells that express MSCF or STO transfected cells (e.g., STO5 or STO8 cells) which express porcine stem cell factor (SCF). Examples of cell culture media that may be utilized in addition to such feeder cells or to supplement such feeder cells can be ES medium (Robertson, 1987; Terato-carcinomas and Embryonic Stem Cells, IRL Press) supplemented with 15% FCS; the ES medium supplemented with growth factors such as leukaemia inhibitory factor (LIF) or LIF plus bFGF. Further, a conditioned media prepared from 5637 carcinoma cell lines may be utilized for initial culturing of PGCs.

Seeded PGC cultures may be maintained at around 37° C. in 5% CO₂ in air. PGCs can be identified by alkaline phosphatase (AP) activity at 1, 3 and 5 days (for example, or at other intervals) using the general procedures set forth above and can also be counted to determine the rate of proliferation. In some cases, the cultured PGCs are trypsinized, rinsed with fresh medium (such as PBS or ES media) and re-passaged every 5 to 10 days (preferably every 6 or 7 days), but significantly older live cultures obtained from PGCs which have stopped proliferating may be transferred to media containing LIF and PSCF (preferably PSCF is provided by feeder cells) and begin to proliferate. The media used for culturing can include a SIRT1 inhibitor, and, for example, porcine SCF and LIF (optionally also including bFGF).

Tissue-Specific Cells. Cells obtained from a tissue, parts of tissue (e.g., parts of organs or whole-organ cultures), or a progenitor cell type that has been committed to a certain cell lineage or has been stimulated to differentiate to a specific cell type can be cultured by the methods described herein, e.g., in the presence of a SIRT1 inhibitor. Tissue cell types include, for example, thymic, lung, liver, brain, muscle, adipocyte, skin, kidney, bone, cartilage, neuronal, gastrointestinal, cardiac, and pancreatic tissue (e.g., pancreatic P cells). Other cell types include bone marrow cells, cardiac muscle cells, dopamine-producing cells, osteoblasts, osteocytes, hepatocytes, fetal brain cells, or myoblasts. Examples of organs include liver, skin, and kidney.

### Cell Culture

In practicing the methods described herein, the cells can be maintained under a variety of conditions, including, e.g., standard cell/tissue culture techniques, such as those specifically suited for a particular cell type. A SIRT1 inhibitor can be added to the cell culture or the SIRT1 inhibitor can be combined with culture media prior to the addition of cells, or both. The SIRT1 inhibitor can be added once or more than once. The exact amount of SIRT1 inhibitor used can vary, e.g., depending on the cell type being cultured (e.g., thymic, lung, liver, brain, muscle, etc.), the status of the cell (e.g., proliferating, quiescent, senescent, pluri-potent, multi-potent, committed to a cell lineage, differentiating, or terminally differentiated). For example, cells can be incubated in humidified chambers at 37° C. at 5-15% CO₂ in media containing bulk ions (e.g., Na⁺, K⁺, Ca²⁺, Mg²⁺, Cl⁻, phosphate, bicarbonate or CO₂); trace elements (e.g., iron, zinc, selenium); sugars (e.g., glucose); amino acids (e.g., 13 essential amino acids; e.g., L-glutamine); vitamins; choline; inositol; serum (e.g., 5-20% heat-inactivated serum; e.g., that contains growth factors); buffering agents; antibiotics (e.g., streptomycin, amphotericin B, penicillin) to control the growth of bacterial and fungal contaminants. Conditioned media can also be used.

The SIRT1 inhibitor can be used (e.g., when used for cells that will be transplanted), e.g., in combination (either sequentially or concomitantly) with other agents used to preserve cells, e.g., a cryoprotectant, dimethyl sulfoxide (DMSO) and antioxidants such as glutathione (e.g., reduced glutathione (GSH)), N-acetyl-L-cysteine (NAC), and members of the lazaroid family of 21-aminosteroids (e.g., U-83836E).

Examples of cell culture media include: Ames Medium; Basal Media Eagle; Click's Medium; Dulbecco's Modified Eagle's Media; Dulbecco's Modified Eagle's Medium/Ham's Nutrient Mixture F-12; Earle's Balanced Salts; Glasgow Minimum Essential Media; Grace's Insect Media; Hanks' Balanced Salts; Iscove's Modified Dulbecco's Media (IMDM); IPL-41 Insect Medium; L-15 Media; M2 and M16 Media; McCoy's 5A Modified Media; MCDB Media; Medium 199; Minimum Essential Medium Eagle (MEM); NCTC Media; Nutrient Mixtures (HAM) F— 10; Nutrient Mixtures (HAM) F-12; Other Salt Mixtures; RPMI-1640 Media; Schneider's Insect Media; Shields and Sang M3 Insect Media; TC-100 Insect Medium; TNM-FH Insect Media; Waymouth Medium MB; William's Medium E. See generally, Mather and Roberts, Introduction to Cell and Tissue Culture: Theory and Technique (Introductory Cell and Molecular Biology Techniques), Kluwer Academic Publishers; 1st edition (1998).

### Cell Transplantation

The cells that have been contacted with a SIRT1 inhibitor can be delivered to a subject, e.g., a subject in need of such cells. In one example, the cells are delivered to a subject having cells that are undergoing normal senescence or quiescence. In another example, the cells are delivered to a subject that has experienced an injury or a disease, such as heart disease, stroke, kidney failure, liver cirrhosis, or macular degeneration. As another example, the cells are delivered to a subject that has experienced a chronically degenerative disease (such as cardiac muscle disease, neurodegenerative disease (e.g., Parkinson's disease, Alzheimer's disease, Huntington's Disease)). As still another example, the cells are delivered to a subject that has experienced a bone disease (e.g., osteoporosis), a blood disease (e.g., a leukemia) or liver disease (e.g., due to alcohol abuse or hepatitis).

The cells can be delivered to a subject that has experienced another condition characterized by unwanted cell loss. As another example, the cells can be delivered to a subject that has undergone an injury, e.g., chemotherapy and/or radiation treatment. As another example, the cells can be delivered to a subject that has suffered a wound, a burn, an ulcer (e.g., ulcer in a diabetic, e.g., diabetic foot ulcer), a surgical wound, a sore, and abrasions. The cells cultured as described herein can be used to repopulate various tissues, such as the liver (Petersen et al., Science 284:1168-1170, 1999) and neuronal tissue (Bjornson et al., Science 283:534-537, 1999). The cells may also serve as a source of cells for various cellular and gene delivery applications.

The cells can be used for grafting, e.g., an autograft or allograft (e.g., HSCs can be used for autologous and allogeneic hematopoietic engraftment). The graft can include e.g., soft tissue (e.g., pedicle grafts, free gingival grafts, subepithelial connective tissue grafts), fetal tissue (e.g., fetal brain tissue), nerve tissue, ovarian tissue, bone tissue, connective tissue, corneal tissue, vascularized tissue, and orthopedic grafts. In another embodiment, the cells can be used for a xenograft (e.g., graft of tissue from one species to a different species, e.g., from a pig to a human). U.S. Pat. No. 6,849,448 describes an exemplary source of cells that is a genetically modified pig, e.g., a pig with an inactivated α-1,3 galactosyltransferase gene.

Alternatively, cells expanded by the methods described herein, e.g., an ex vivo (e.g., in vitro) expanded HSC composition, may be used for gene therapy to treat any of a number of diseases. In such cases, cells containing a gene that is absent or mutated in the subject or containing a transgene of interest directed toward a particular disease target are prepared in vitro and re-infused into a subject, e.g., such that the cell type(s) targeted by the disease are repopulated by differentiation of the cells in the HSC composition following re-infusion into the subject. The cells can also be genetically modified using gene therapy techniques (see below) to express a desired gene. The modified cells can then be transplanted into a subject, e.g., for the treatment of disease or injury by any method that is appropriate for the type of cells being transplanted and the transplant site. The cells can be transplanted intravenously, or they can be transplanted directly at a target site, e.g., the site of injury or disease. Alternatively, the cells can be maintained in culture or cryopreserved for future studies or transplantation.

In addition, culturing of a subject's own cells, e.g., bone marrow or HSCs (e.g., bone marrow extracted from the subject before commencing chemotherapy or radiation therapy) in the presence of a SIRT1 inhibitor can be useful, e.g., to obtain an expanded population of cells and to avoid the current need for immune suppression by minimizing the potential for GVHD following transplantation.

In another embodiment, the cells from another source (e.g., a donor of the same species (e.g., an HLA-matched donor) or of a different species (e.g, a xenotransplant, e.g., from a transgenic pig)) can be transplanted into the subject in need of such transplantation, maintained in culture, or cryopreserved for future use or studies.

In another embodiment, the cells cultured by the methods described herein may contain an endogenous gene that is absent from or mutated in cells of the recipient who would receive such cells. These cells can be transplanted to a subject to restore the function provided by the gene that is absent or mutated in the subject.

Methods of introduction of cells for transplantation include but are not limited to intradermal, intramuscular, intraperitoneal, intravenous, subcutaneous, intranasal, and epidural routes. The cells cultured as described herein may be administered by any convenient route, for example by infusion or bolus injection, by absorption through epithelial or mucocutaneous linings (e.g., oral mucosa, rectal and intestinal mucosa) and may be administered together with other biologically active agents. Administration can be systemic or local (e.g., into tissue, e.g., liver, muscle, brain, pancreas).

The cells cultured as described herein can be derived from the subject to which it is administered, i.e., the transplant is autologous. Alternatively, the cells cultured as described herein can be derived from a heterologous source, e.g., a cell donor (e.g., a blood or platelet donor, a tissue donor, a biopsy sample) or from a primary or established cell line, e.g., a progenitor cell line, e.g., in a pluri-potent or multi-potent state or that has been treated so as to differentiate in a committed cell type lineage.

Cells cultured by the methods described herein can be maintained in culture and not transferred to a subject. Such cells can be maintained in culture by the methods described herein to provide a source of cells that can be administered to a subject in the future, or these cells can be used for in vitro studies, e.g., for the characterization of the effects of a compounds or drug on these cells, e.g., the toxicity of a compound and drug or the responsiveness of such cells to a compound or drug. The cells can be cryopreserved for future use.

A SIRT1 inhibitor can also be administered to a transplant recipient, e.g., a subject who has received transplanted cells, e.g., to maintain or extend the capacity of transplanted precursor cells while in the subject. The SIRT1 inhibitor can be provided as a pharmaceutical composition, e.g., subsequent to transplantation, e.g., for a limited or prolonged duration, e.g., for less than two weeks or less than one month.

A SIRT1 inhibitor can be administered to a transplant donor, e.g., a subject who is donating cells for transplantation (e.g., autologous or heterologous), e.g., to extend or maintain the capacity of the cells to be transplanted prior to donation and/or to maximize the cell harvest obtained from the donor. The SIRT1 inhibitor can be provided as a pharmaceutical composition, e.g., prior to transplantation, e.g., for a limited or prolonged duration, e.g., for less than two weeks or less than one month, prior to donation.

### Gene Delivery

Gene delivery encompasses providing an exogenous gene to a cell, e.g., for gene correction therapy and transfer of therapeutic genes, e.g., to treat cancer, infectious diseases, monogenic diseases, multigenic diseases, hereditary diseases, and acquired diseases. The gene can be delivered, e.g., in vitro, e.g., to a cell that has been, is, or will be cultured with a SIRT1 inhibitor.

Exemplary disease targets include, but are not limited to cancer, such as prostate cancer, breast cancer, lung cancer, colorectal cancer, melanoma and leukemia; infectious diseases, such as HIV, monogenic diseases such as CF, hemophilia, phenylketonuria, ADA, familial hypercholesterolemia, and multigenic diseases, such as restenosis, ischemia, and diabetes; degenerative diseases such as neurodegenerative diseases such as Parkinson's disease, Alzheimer's disease, and Huntington's Disease. See, e.g., Blaese et al., Science 270:475-480, 1995; Wingo et al., Cancer 82:1197-1207, 1998.

Cell transduction is possible in vivo. However, it is simpler and more easily controlled ex vivo. When cells are cultured ex vivo, they can be cultured in the presence of a SIRT1 inhibitor, e.g., such culturing can be performed with HSCs, useful for therapeutic gene therapy (see, e.g., Buetler, Biol. Blood Marrow Transplant. 5:273-276, 1999; Dao, Leukemia 13:1473-1480, 1999; and see generally Morgan et al., Ann. Rev. Biochem. 62:191-217, 1993; Culver et al., Trends Genet. 10:174-178, 1994; and U.S. Pat. No. 5,399,346).

A therapeutic gene therapy regimen may include one or more of: obtaining cells from a subject, enriching or purifying the cells of interest, culturing, e.g., expanding, the cells by the methods described herein, introducing the gene of interest into the cells, and reintroducing the cells into the subject. The gene can be contained within a vector. The gene can be introduced, e.g., by transfection or viral transduction.

The cells cultured by the methods described herein can be used as recipients for gene delivery. The nucleic acid introduced into the cells may encode any desired protein, e.g., a protein missing or dysfunctional in a disease or disorder. For general reviews of the methods of gene therapy, see Goldspiel et al., Clinical Pharmacy 12:488-505, 1993; Wu and Wu, Biotherapy 3:87-95, 1991; Tolstoshev, Ann. Rev. Pharmacol. Toxicol. 32:573-596, 1993; Mulligan, Science 260:926-932, 1993; and Morgan and Anderson, Ann. Rev. Biochem. 62:191-217, 1993. Methods commonly known in the art of recombinant DNA technology that can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

A gene whose expression is desired in a subject can be introduced into the cells such that it is expressible by the cells and/or their progeny, and these cells are then administered in vivo for therapeutic effect. Cells cultured by the methods described herein can be used in any appropriate method of gene therapy. The resulting action of the cells cultured by the methods described herein and carrying a transgene administered to a subject can, for example, lead to the activation or inhibition of a pre-selected gene in the subject, or can provide a gene product that is absent or at low levels in the subject, thus leading to improvement of the diseased condition afflicting the subject.

Alternatively, cells cultured by the methods described herein into which a gene has been introduced can be maintained in culture and not transferred to a subject. Such cells can be maintained in culture by the methods described herein to provide a source of cells that can be administered to a subject in the future, or these cells can be used for in vitro studies, e.g., for the characterization of the effects of a compounds or drug on these cells, e.g., the toxicity of a compound and drug or the responsiveness of such cells to a compound or drug. The cells can also be cryopreserved for future use.

One common method of practicing gene therapy uses viral vectors, for example retroviral vectors (see Miller et al., Meth. Enzymol. 217:581-599, 1993; Boesen et al., Biotherapy 6:291-302, 1994; Clowes et al., J. Clin. Invest. 93:644-651, 1994; Kiem et al., Blood 83:1467-1473, 1994; Salmons and Gunzberg, Human Gene Therapy 4:129-141, 1993; and Grossman and Wilson, Curr. Opin. in Genetics and Devel. 3:110-114, 1993), adenovirus vectors (Kozarsky and Wilson, Current Opinion in Genetics and Development 3:499-503, 1993; Rosenfeld et al., Science 252:431-434, 1991; Rosenfeld et al., Cell 68:143-155, 1992; and Mastrangeli et al., J. Clin. Invest. 91:225-234, 1993), adenovirus-associated vectors (AAV; see, for example, Walsh et al., Proc. Soc. Exp. Biol. Med. 204:289-300, 1993), adenovirus vectors, lentivirus vectors, herpes virus vectors, pox virus vectors; non-viral vectors, for example, naked DNA delivered via liposomes, receptor-mediated delivery, calcium phosphate transfection, lipofection, electroporation, particle bombardment (e.g., gene gun), microinjection, cell fusion, chromosome-mediated gene transfer, microcell-mediated gene transfer, spheroplast fusion, or pressure-mediated gene delivery. Numerous techniques can be used for the introduction of foreign genes into cells (see e.g., Loeffler and Behr, Meth. Enzymol. 217:599-618, 1993; Cohen et al., Meth. Enzymol. 217:618-644, 1993; Cline, Pharmac. Ther. 29:69-92, 1985) and may be used in accordance with cells cultured by the methods described herein. The method of transfer can include the transfer of a selectable marker to the cells. The cells are then placed under selection to isolate those cells that have taken up and are expressing the transferred gene. Those cells are then delivered to a subject, maintained in culture, or cryopreserved for future use. The technique should provide for the stable transfer of the gene to the cell, so that the gene is expressible by the cell and preferably heritable and expressible by its cell progeny.

A desired gene can also be introduced intracellularly and incorporated within host precursor cell DNA for expression, e.g., by homologous recombination (Koller and Smithies, Proc. Natl. Acad. Sci. USA 86:8932-8935, 1989; Zijlstra et al., Nature 342:435-438, 1989).

### SIRT1 Inhibitors

The SIRT1 inhibitors can be prepared as purified preparations, e.g., 95%, 96%, 97%, 98%, 99%, or 100% pure. The SIRT1 inhibitors can be an active ingredient in a composition (e.g., liquid or solid, e.g., a solid that is added to the cell culture as a solid or is reconstituted (e.g., with a sterile buffer or sterile saline) prior to use. The SIRT1 inhibitors can be in a preparation that contains carrier ingredients, e.g., such as salts (e.g., pharmaceutically-acceptable salts), buffers, and/or stabilizers. A particular SIRT1 inhibitor can be present in the composition, e.g., between 0.1-90% (w/w), e.g., 1-30% (w/w).

Examples of SIRT1 inhibitors include those compounds in one of the three classes described below.

One class of compounds that can be used as a SIRT1 inhibitor has a general formula (I) and contains a substituted pentacyclic or hexacyclic core containing one or two, respectively, oxygen, nitrogen, or sulfur atoms as a constituent atom of the ring, e.g., X and Y in formula (I) below.

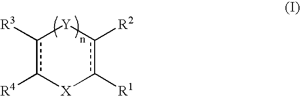

Any ring carbon atom can be substituted. For example, R¹, R², R³, and R⁴ may include without limitation substituted or unsubstituted alkyl, cycloalkyl, alkenyl, alkynyl, heterocyclyl, heterocycloalkenyl, cycloalkenyl, aryl, heteroaryl, etc. The pentacyclic or hexacyclic core may be saturated, i.e. containing no double bonds, or partially or fully saturated, i.e. one or two double bonds respectively. When n=0, “X” may be oxygen, sulfur, or nitrogen, e.g., NR⁷. The substituent R⁷ can be without limitation hydrogen, alkyl, e.g., C1, C2, C3, C4 alkyl, SO₂(aryl), acyl, or the ring nitrogen may form part of a carbamate, or urea group. When n=1, X can be NR⁷, O, or S; and Y can be NR^{7′}, O or S. X and Y can be any combination of heteroatoms, e.g., N,N, N,O, N, S, etc.

A preferred subset of compounds of formula (I) includes those having one, or preferably, two rings that are fused to the pentacyclic or hexacyclic core, e.g., R¹ and R², together with the carbons to which they are attached, and/or R³ and R⁴, together with the carbons to which they are attached, can form, e.g., C₅-C₁₀ cycloalkyl (e.g., C5, C6, or C7), C₅-C₁₀ heterocyclyl (e.g., C5, C6, or C7), C₅-C₁₀ cycloalkenyl (e.g., C5, C6, or C7), C₅-C₁₀ heterocycloalkenyl (e.g., C5, C6, or C7), C₆-C₁₀ aryl (e.g., C6, C8 or C10), or C₆-C₁₀ heteroaryl (e.g., C5 or C6). Fused ring combinations may include without limitation one or more of the following:

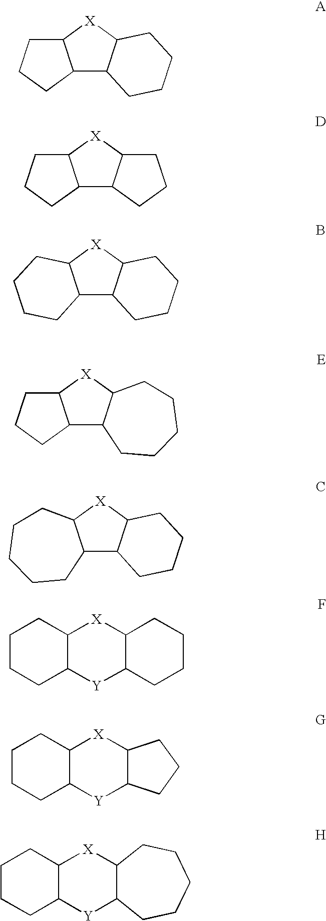

Preferred combinations include B, e.g. having C₆ aryl and C₆ cycloalkenyl (B1), and C, e.g. having C₆ aryl and C₇ cycloalkenyl (C1):

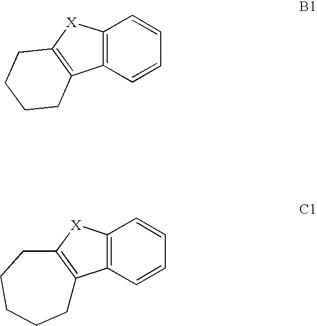

Each of these fused ring systems may be optionally substituted with substitutents, which may include without limitation halo, hydroxy, C₁-C₁₀ alkyl (C1, C2, C3, C4, C5, C6, C7, C8, C9, C10), C₁-C₆ haloalkyl (C1, C2, C3, C4, C5, C6), C₁-C₁₀ alkoxy (C1, C2, C3, C4, C5, C6, C7, C8, C9, C10), C₁-C₆ haloalkoxy (C1, C2, C3, C4, C5, C6), C₆-C₁₀ aryl (C6, C7, C8, C9, C10), C₅-C₁₀ heteroaryl (C5, C6, C7, C8, C9, C10), C₇-C₁₂ aralkyl (C7, C8, C9, C10, C11, C12), C₇-C₁₂ heteroaralkyl (C7, C8, C9, C10, C11, C12), C₃-C₈ heterocyclyl (C3, C4, C5, C6, C7, C8), C₂-C₁₂ alkenyl (C2, C3, C4, C5, C6, C7, C8, C9, C10, C11, C12), C₂-C₁₂ alkynyl (C2, C3, C4, C5, C6, C7, C8, C9, C10, C11, C12), C₅-C₁₀ cycloalkenyl (C5, C6, C7, C8, C9, C10), C₅-C₁₀ heterocycloalkenyl (C5, C6, C7, C8, C9, C10), carboxy, carboxylate, cyano, nitro, amino, C₁-C₆ alkyl amino (C1, C2, C3, C4, C5, C6), C₁-C₆ dialkyl amino (C1, C2, C3, C4, C5, C6), mercapto, SO₃H, sulfate, S(O)NH₂, S(O)₂NH₂, phosphate, C₁-C₄ alkylenedioxy (C1, C2, C3, C4), oxo, acyl, aminocarbonyl, C₁-C₆ alkyl aminocarbonyl (C1, C2, C3, C4, C5, C6), C₁-C₆ dialkyl aminocarbonyl (C1, C2, C3, C4, C5, C6), C₁-C₁₀ alkoxycarbonyl (C1, C2, C3, C4, C5, C6, C7, C8, C9, C10), C₁-C₁₀ thioalkoxycarbonyl (C1, C2, C3, C4, C5, C6, C7, C8, C9, C10), hydrazinocarbonyl, C₁-C₆ alkyl hydrazinocarbonyl (C1, C2, C3, C4, C5, C6), C₁-C₆ dialkyl hydrazinocarbonyl (C1, C2, C3, C4, C5, C6), hydroxyaminocarbonyl, etc. Preferred substituents include halo (e.g., fluoro, chloro, bromo), C₁-C₁₀ alkyl (e.g., C1, C2, C3, C4, C5, C6, C7, C8, C9, C10), C₁-C₆ haloalkyl (e.g., C1, C2, C3, C4, C5, C6, e.g., CF₃), C₁-C₆ haloalkoxyl (e.g., C1, C2, C3, C4, C5, C6, e.g., OCF₃), or aminocarbonyl. The substitution pattern on the two fused rings may be selected as desired, e.g., one ring may be substituted and the other is not, or both rings may be substituted with 1-5 substitutents (1,2,3,4,5 substitutents). The number of substituents on each ring may be the same or different. Preferred substitution patterns are shown below:

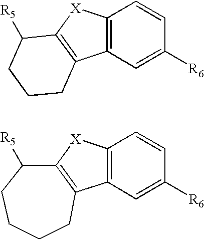

In certain embodiments, when n is 0 and X is NR⁷, the nitrogen substituent R⁷ can form a cyclic structure with one of the fused rings containing, e.g., 4-6 carbons, 1-3 nitrogens, 0-2 oxygens and 0-2 sulfurs. This cyclic structure may optionally be substituted with oxo or C₁-C₆ alkyl.

Combinations of substituents and variables include those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

TABLE 1
Exemplary compounds

| Compound | | Ave. SirT1 p53-382 |
|---|---|---|
| number | Chemical name | IC50 (μM) |
| 1 | 7-Chloro-1,2,3,4-tetrahydro-cyclopenta[b]indole-3-carboxylic | A |
| | acid amide | |
| 2 | 2,3,4,9-Tetrahydro-1H-b-carboline-3-carboxylic acid amide | C |
| 3 | 6-Bromo-2,3,4,9-tetrahydro-1H-carbazole-2-carboxylic acid | B |
| | amide | |
| 4 | 6-Methyl-2,3,4,9-tetrahydro-1H-carbazole-1-carboxylic acid | A |
| | amide | |
| 5 | 2,3,4,9-Tetrahydro-1H-carbazole-1-carboxylic acid amide | B |
| 6 | 2-Chloro-5,6,7,8,9,10-hexahydro-cyclohepta[b]indole-6- | A |
| | carboxylic acid amide | |
| 7 | 6-Chloro-2,3,4,9-tetrahydro-1H-carbazole-1-carboxylic acid | C |
| | hydroxyamide | |
| 8 | 6-Chloro-2,3,4,9-tetrahydro-1H-carbazole-1-carboxylic acid | A |
| | amide | |
| 9 | 6-Chloro-2,3,4,9-tetrahydro-1H-carbazole-2-carboxylic acid | C |
| | amide | |
| 10 | 1,2,3,4-Tetrahydro-cyclopenta[b]indole-3-carboxylic acid | B |
| | amide | |
| 11 | 6-Chloro-2,3,4,9-tetrahydro-1H-carbazole-1-carboxylic acid (5- | B |
| | chloro-pyridin-2-yl)-amide | |
| 12 | 1,6-Dimethyl-2,3,4,9-tetrahydro-1H-carbazole-1-carboxylic | C |
| | acid amide | |
| 13 | 6-Trifluoromethoxy-2,3,4,9-tetrahydro-1H-carbazole-2- | C |
| | carboxylic acid amide | |
| 14 | 6-Chloro-2,3,4,9-tetrahydro-1H-carbazole-1-carboxylic acid | D |
| | diethylamide | |
| 15 | 6-Chloro-2,3,4,9-tetrahydro-1H-carbazole-1-carboxylic acid | D |
| | carbamoylmethyl-amide | |
| 16 | 8-Carbamoyl-6,7,8,9-tetrahydro-5H-carbazole-1-carboxylic | D |
| | acid | |
| 17 | 6-Methyl-2,3,4,9-tetrahydro-1H-carbazole-1-carboxylic acid | D |
| 18 | 8-Carbamoyl-2,3,4,9-tetrahydro-1H-carbazole-1-carboxylic | D |
| | acid ethyl ester | |
| 19 | [(6-Chloro-2,3,4,9-tetrahydro-1H-carbazole-1-carbonyl)- | D |
| | amino]-acetic acid ethyl ester | |
| 20 | 9-Benzyl-2,3,4,9-tetrahydro-1H-carbazole-1-carboxylic acid | D |
| | amide | |
| 21 | 6-Chloro-2,3,4,9-tetrahydro-1H-carbazole-1-carboxylic acid | D |
| | methyl ester | |
| 22 | 6-Chloro-2,3,4,9-tetrahydro-1H-carbazole-1-carboxylic acid | D |
| 23 | C-(6-Methyl-2,3,4,9-tetrahydro-1H-carbazol-1-yl)-methylamine | D |
| 24 | 6,9-Dimethyl-2,3,4,9-tetrahydro-1H-carbazole-1-carboxylic | D |
| | acid amide | |
| 25 | 7-Methyl-1,2,3,4-tetrahydro-cyclopenta[b]indole-3-carboxylic | D |
| | acid amide | |
| 26 | 6-Chloro-2,3,4,9-tetrahydro-1H-carbazole-1-carboxylic acid | D |
| | ethylamide | |
| 27 | 2-(1-Benzyl-3-methylsulfanyl-1H-indol-2-yl)-N-p-tolyl- | D |
| | acetamide | |
| 28 | N-Benzyl-2-(1-methyl-3-phenylsulfanyl-1H-indol-2-yl)- | D |
| | acetamide | |
| 29 | N-(4-Chloro-phenyl)-2-(1-methyl-3-phenylsulfanyl-1H-indol-2- | D |
| | yl)-acetamide | |
| 30 | N-(3-Hydroxy-propyl)-2-(1-methyl-3-phenylsulfanyl-1H-indol-2- | D |
| | yl)-acetamide | |
| 31 | 2-(1-Benzyl-3-phenylsulfanyl-1H-indol-2-yl)-N-(3-hydroxy- | D |
| | propyl)-acetamide | |
| 32 | 2-(1-Benzyl-3-methylsulfanyl-1H-indol-2-yl)-N-(4-methoxy- | D |
| | phenyl)-acetamide | |
| 33 | 2-(1-Benzyl-1H-indol-2-yl)-N-(4-methoxy-phenyl)-acetamide | D |
| 34 | 2-(1-Methyl-3-methylsulfanyl-1H-indol-2-yl)-N-p-tolyl- | D |
| | acetamide | |
| 35 | 2-(1-Benzyl-3-methylsulfanyl-1H-indol-2-yl)-N-(2-chloro- | D |
| | phenyl)-acetamide | |
| 36 | 2-(1,5-Dimethyl-3-methylsulfanyl-1H-indol-2-yl)-N-(2-hydroxy- | D |
| | ethyl)-acetamide | |
| 37 | (6-Chloro-2,3,4,9-tetrahydro-1H-carbazol-1-yl)-[4-(furan-2- | D |
| | carbonyl)-piperazin-1-yl]-methanone | |
| 38 | 2-(1-Benzyl-1H-indol-2-yl)-N-(2-chloro-phenyl)-acetamide | D |
| 39 | 6-Chloro-2,3,4,9-tetrahydro-1H-carbazole-1-carboxylic acid | D |
| | ethyl ester | |
| 40 | 6-Chloro-9-methyl-2,3,4,9-tetrahydro-1H-carbazole-4- | D |
| | carboxylic acid ethyl ester | |
| 41 | 5,7-Dichloro-2,3,4,9-tetrahydro-1H-carbazole-1-carboxylic acid | D |
| | ethyl ester | |
| 42 | 7-Chloro-2,3,4,9-tetrahydro-1H-carbazole-1-carboxylic acid | D |
| | ethyl ester | |
| 43 | 5,7-Dichloro-2,3,4,9-tetrahydro-1H-carbazole-1-carboxylic acid | D |
| 44 | 6-Chloro-9-methyl-2,3,4,9-tetrahydro-1H-carbazole-4- | D |
| | carboxylic acid | |
| 45 | 6-Chloro-9-methyl-2,3,4,9-tetrahydro-1H-carbazole-4- | D |
| | carboxylic acid amide | |
| 46 | 6-Morpholin-4-yl-2,3,4,9-tetrahydro-1H-carbazole-1-carboxylic | D |
| | acid ethyl ester | |
| 47 | 6-Morpholin-4-yl-2,3,4,9-tetrahydro-1H-carbazole-1-carboxylic | D |
| | acid amide | |
| 48 | 6-Bromo-2,3,4,9-tetrahydro-1H-carbazole-1-carboxylic acid | D |
| | ethyl ester | |
| 49 | 6-Fluoro-2,3,4,9-tetrahydro-1H-carbazole-1-carboxylic acid | D |
| | ethyl ester | |
| 50 | 3-Carbamoyl-1,3,4,9-tetrahydro-b-carboline-2-carboxylic acid | D |
| | tert-butyl ester | |
| 51 | 6-Chloro-2,3,4,9-tetrahydro-1H-carbazole-1-carboxylic acid (1- | D |
| | phenyl-ethyl)-amide | |
| 52 | 7,8-Difluoro-2,3,4,9-tetrahydro-1H-carbazole-1-carboxylic acid | D |
| | amide | |
| 53 | 6-bromo-2,3,4,9-tetrahydro-1H-carbazole-1-carboxylic acid | D |
| 54 | 6-hydroxy-2,3,4,9-tetrahydro-1H-carbazole-1-carboxylic acid | C |
| 55 | 6-bromo-2,3,4,9-tetrahydro-1H-carbazole-2-carboxamide | B |
| 56 | 6-chloro-2,3,4,9-tetrahydro-1H-pyrido[3,4-b]indole-1- | C |
| | carboxamide | |
| 57 | 6-bromo-2,3,4,9-tetrahydro-1H-pyrido[3,4-b]indole-1- | D |
| | carboxamide | |
| 58 | 2-acetyl-6-chloro-2,3,4,9-tetrahydro-1H-pyrido[3,4-b]indole-1- | C |
| | carboxamide | |

*Compounds having activity designated with an A have an IC₅₀ of less than 1.0 μM. Compounds having activity designated with a B have an IC₅₀ between 1.0 μM and 10.0 μM. Compounds having activity designated with a C have an IC₅₀ greater than 10.0 μM. Compounds designated with a D were not tested in this assay.

The synthesis and calculations of the IC₅₀ for these compounds are described in U.S. patent application Ser. No. 10/940,269 (filed on Sep. 13, 2004) and U.S. patent application Ser. No. 11/077,664 (filed on Mar. 11, 2005).

Other classes of compounds that can be used as a SIRT1 inhibitor have a general formula (XXIII), (XXIV), (XXV), or (XXVI) and contain a substituted cyclic (e.g., pentacyclic or hexacyclic) or polycyclic core containing one or more oxygen, nitrogen, or sulfur atoms as a constituent atom of the ring(s).

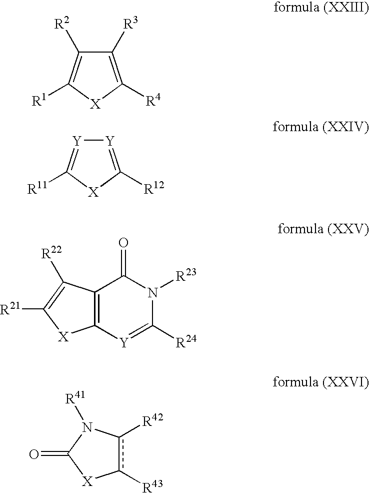

Any ring carbon atom can be substituted. The cyclic or polycyclic core may be partially or fully saturated, i.e. one or two double bonds respectively.

A preferred subset of compounds of formula (XXIII) includes those having a ring that is fused to the pentacyclic core, e.g., R¹ and R², together with the carbons to which they are attached, and/or R³ and R⁴, together with the carbons to which they are attached, form C₅-C₁₀ cycloalkenyl (e.g., C5, C6, or C7), C₅-C₁₀ heterocycloalkenyl (e.g., C5, C6, or C7), C₆-C₁₀ aryl (e.g., C6, C8 or C10), or C₆-C₁₀ heteroaryl (e.g., C5 or C6). Fused ring combinations may include without limitation one or more of the following:

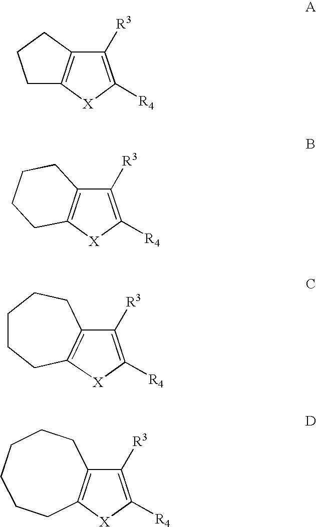

Each of these fused ring systems may be optionally substituted with substitutents, which may include without limitation halo, hydroxy, C₁-C₁₀ alkyl (C1, C2, C3, C4, C5, C6, C7, C8, C9, C10), C₁-C₆ haloalkyl (C1, C2, C3, C4, C5, C6), C₁-C₁₀ alkoxy (C1, C2, C3, C4, C5, C6, C7, C8, C9, C10), C₁-C₆ haloalkoxy (C1, C2, C3, C4, C5, C6), C₆-C₁₀ aryl (C6, C7, C8, C9, C10), C₅-C₁₀ heteroaryl (C5, C6, C7, C8, C9, C10), C₇-C₁₂ aralkyl (C7, C8, C9, C10, C11, C12), C₇-C₁₂ heteroaralkyl (C7, C8, C9, C10, C11, C₁₂), C₃-C₈ heterocyclyl (C3, C4, C5, C6, C7, C8), C₂-C₁₂ alkenyl (C2, C3, C4, C5, C6, C7, C8, C9, C10, C11, C12), C₂-C₁₂ alkynyl (C2, C3, C4, C5, C6, C7, C8, C9, C10, C11, C12), C₅-C₁₀ cycloalkenyl (C5, C6, C7, C8, C9, C10), C₅-C₁₀ heterocycloalkenyl (C5, C6, C7, C8, C9, C10), carboxy, carboxylate, cyano, nitro, amino, C₁-C₆ alkyl amino (C1, C2, C3, C4, C5, C6), C₁-C₆ dialkyl amino (C1, C2, C3, C4, C5, C6), mercapto, SO₃H, sulfate, S(O)NH₂, S(O)₂NH₂, phosphate, C₁-C₄ alkylenedioxy (C1, C2, C3, C4), oxo, acyl, aminocarbonyl, C₁-C₆ alkyl aminocarbonyl (C1, C2, C3, C4, C5, C6), C₁-C₆ dialkyl aminocarbonyl (C1, C2, C3, C4, C5, C6), C₁-C₁₀ alkoxycarbonyl (C1, C2, C3, C4, C5, C6, C7, C8, C9, C10), C₁-C₁₀ thioalkoxycarbonyl (C1, C2, C3, C4, C5, C6, C7, C8, C9, C10), hydrazinocarbonyl, C₁-C₆ alkyl hydrazinocarbonyl (C1, C2, C3, C4, C5, C6), C₁-C₆ dialkyl hydrazinocarbonyl (C1, C2, C3, C4, C5, C6), hydroxyaminocarbonyl, etc. Preferred substituents include C₁-C₁₀ alkyl (e.g., C1, C2, C3, C4, C5, C6, C7, C8, C9, C10), aminocarbonyl, and amido. The substitution pattern can be selected as desired.

Another preferred subset of compounds of formula (XXIII) includes those where R¹ and R² are C₁-C₆ alkyl (e.g., wherein R¹ and R² are both CH₃).

In still another preferred subset of the compounds of formula (XXIII), R³ is a substituted or unsubstituted aminocarbonyl and R⁴ is an amido substituted with a substituent.

In still another preferred subset of the compounds of formula (XXIII), X is S.

A preferred subset of compounds of formula (XXIV) includes those having a triazole core (i.e., wherein X is NR¹⁶ and both Ys are N).

Another preferred subset of compounds include those where R¹¹ is a substituted thioalkoxy. Where R¹¹ is thioalkoxy, preferred substituents include amino carbonyl. An example of a preferred subset is provided below.

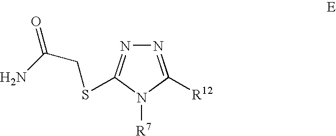

Still another subset of preferred embodiments include those where R¹² is aryl, arylalkyl, heteroaryl, heteroarylalkyl, and alkyl substituted with heteroaryloxy or aryloxy. Each aryl and heteroaryl is optionally substituted.

Still another subset of preferred embodiments include those wherein X is NR⁷ and R⁷ is aryl, heteroaryl, arylalkyl or heteroarylalkyl, each is which is optionally substituted.

A preferred subset of compounds of formula (XXV) includes those having one of the following polycyclic cores:

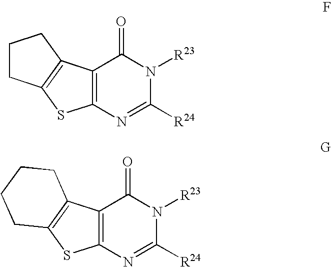

The polycyclic core can be substituted with one or more suitable substituents.

A preferred subset of compounds of formula (XXVI) includes those having the following polycyclic core:

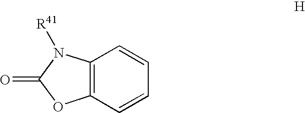

The polycyclic core can be substituted with one or more suitable substituents.

Other examples of embodiments are depicted in the following structures below together with representative examples of Sir2 activity.

TABLE 2
Activity of Triazoles (conc. in μM)

| Compound | | | |
|---|---|---|---|
| Number | Chemical Name | SirT1 (μM) | SirT2 (μM) |
| 1 | 2-[4-Benzyl-5-(1H-indol-3-ylmethyl)-4H- | B | C |
| | [1,2,4]triazol-3-ylsulfanyl]-acetamide | | |
| 2 | 2-[4-(4-Methoxy-phenyl)-5-(naphthalen-1- | B | C |
| | yloxymethyl)-4H-[1,2,4]triazol-3-ylsulfanyl]- | | |
| | acetamide | | |
| 3 | 2-(5-Benzyl-4-p-tolyl-4H-[1,2,4]triazol-3- | B | C |
| | ylsulfanyl)-acetamide | | |
| 4 | 2-[5-(2-Bromo-phenyl)-4-p-tolyl-4H- | C | B |
| | [1,2,4]triazol-3-ylsulfanyl]-acetamide | | |

TABLE 3
Activity of representative compounds (conc. in μM)

| Compound | | | |
|---|---|---|---|
| Number | Chemical Name | SirT1 (μM) | SirT2 (μM) |
| 5 | (5-Cyclohexyl-4-oxo-2,3,4,5-tetrahydro-1H- | B | C |
| | 8-thia-5,7-diaza-cyclopenta[a]inden-6- | | |
| | ylsulfanyl)-acetic acid | | |
| 6 | 2-(6-Bromo-2-oxo-benzooxazol-3-yl)- | B | C |
| | acetamide | | |
| 7 | 3-(3-Amino-4-oxo-3,4,5,6,7,8-hexahydro- | C | C |
| | benzo[4,5]thieno[2,3-d]pyrimidin-2-yl)- | | |
| | propionic acid | | |

TABLE 4
Activity of representative compounds

| | | SirT1 |
|---|---|---|
| Compound | | p53-382- |
| Number | Chemical Name | FdL IC50 |
| 8 | 3-Chloro-benzo[b]thiophene-2-carboxylic acid | D |
| | carbamoylmethyl ester | |
| 9 | 4,5-Dimethyl-2-[2-(5-methyl-3-nitro-pyrazol-1- | C |
| | yl)-acetylamino]-thiophene-3-carboxylic acid | |
| | amide | |
| 10 | Furan-2-carboxylic acid (3-carbamoyl-4,5,6,7- | D |
| | tetrahydro-benzo[b]thiophen-2-yl)-amide | |
| 11 | 5-Bromo-furan-2-carboxylic acid (3- | C |
| | carbamoyl-4,5-dimethyl-thiophen-2-yl)-amide | |
| 12 | 2-[(Thiophene-2-carbonyl)-amino]-4,5,6,7- | D |
| | tetrahydro-benzo[b]thiophene-3-carboxylic acid | |
| | amide | |
| 13 | Furan-2-carboxylic acid (3-carbamoyl-5,6- | D |
| | dihydro-4H-cyclopenta[b]thiophen-2-yl)-amide | |
| 14 | Tetrahydro-furan-2-carboxylic acid (3- | D |
| | carbamoyl-6-methyl-4,5,6,7-tetrahydro- | |
| | benzo[b]thiophen-2-yl)- | |
| | amide | |
| 15 | Tetrahydro-furan-2-carboxylic acid (3- | C |
| | carbamoyl-4,5-dimethyl-thiophen-2-yl)-amide | |
| 16 | 2-(3,4-Dichloro-benzoylamino)-6-methyl- | D |
| | 4,5,6,7-tetrahydro-benzo[b]thiophene-3- | |
| | carboxylic acid amide | |
| 17 | 2-[2-(3-Nitro-[1,2,4]triazol-1-yl)-acetylamino]- | D |
| | 4,5,6,7-tetrahydro-benzo[b]thiophene-3- | |
| | carboxylic | |
| | acid amide | |
| 18 | 2-(4-Fluoro-benzoylamino)-4,5-dimethyl- | D |
| | thiophene-3-carboxylic acid amide | |
| 19 | 2-(3-Chloro-benzoylamino)-4,5,6,7-tetrahydro- | D |
| | benzo[b]thiophene-3-carboxylic acid amide | |
| 20 | Pyrazine-2-carboxylic acid (3-carbamoyl- | D |
| | 4,5,6,7-tetrahydro-benzo[b]thiophen-2-yl)- | |
| | amide | |
| 21 | 3-Chloro-benzo[b]thiophene-2-carboxylic acid | D |
| | (3-carbamoyl-4,5-dimethyl-thiophen-2-yl)- | |
| | amide | |
| 22 | 5-Bromo-N-(3-carbamoyl-4,5,6,7-tetrahydro- | D |
| | benzo[b]thiophen-2-yl)-nicotinamide | |
| 23 | 4-Bromo-1-methyl-1H-pyrazole-3-carboxylic | D |
| | acid (3-carbamoyl-5,6-dihydro-4H- | |
| | cyclopenta[b]thiophen- | |
| | 2-yl)-amide | |
| 24 | 5-Bromo-furan-2-carboxylic acid (3- | D |
| | carbamoyl-4,5,6,7-tetrahydro- | |
| | benzo[b]thiophen-2-yl)-amide | |
| 25 | 2-(3,4-Dichloro-benzoylamino)-4,5,6,7- | D |
| | tetrahydro-benzo[b]thiophene-3-carboxylic acid | |
| | amide | |
| 26 | 2-(Cyclopropanecarbonyl-amino)-4,5- | C |
| | dimethyl-thiophene-3-carboxylic acid amide | |
| 27 | 2-(Cyclohexanecarbonyl-amino)-4,5,6,7- | D |
| | tetrahydro-benzo[b]thiophene-3-carboxylic acid | |
| | amide | |
| 28 | 2-(2,5-Dichloro-benzoylamino)-4,5-dimethyl- | D |
| | thiophene-3-carboxylic acid amide | |
| 29 | N-(3-Carbamoyl-4,5-dimethyl-thiophen-2-yl)- | C |
| | isonicotinamide | |
| 30 | Pyrazine-2-carboxylic acid (3-carbamoyl-4,5- | C |
| | dimethyl-thiophen-2-yl)-amide | |
| 31 | 2-(5-Pyridin-4-yl-2H-[1,2,4]triazol-3-yl)- | D |
| | acetamide | |
| 32 | 2-(Cyclopentanecarbonyl-amino)-6-methyl- | A |
| | 4,5,6,7-tetrahydro-benzo[b]thiophene-3- | |
| | carboxylic acid amide | |
| 33 | 2-(3-Methyl-butyrylamino)-4,5,6,7,8,9- | C |
| | hexahydro-cycloocta[b]thiophene-3-carboxylic | |
| | acid amide | |
| 34 | 2-(Cyclopropanecarbonyl-amino)-5,6,7,8- | C |
| | tetrahydro-4H-cyclohepta[b]thiophene-3- | |
| | carboxylic acid amide | |
| 35 | 6-Methyl-2-propionylamino-4,5,6,7-tetrahydro- | B |
| | benzo[b]thiophene-3-carboxylic acid amide | |
| 36 | 2-Amino-6-methyl-4,5,6,7-tetrahydro- | C |
| | benzo[b]thiophene-3-carboxylic acid amide | |
| 37 | 2-Amino-5-phenyl-thiophene-3-carboxylic acid | C |
| | amide | |
| 38 | 2-Amino-6-ethyl-4,5,6,7-tetrahydro- | C |
| | benzo[b]thiophene-3-carboxylic acid amide | |
| 39 | 2-(1-Benzyl-3-methylsulfanyl-1H-indol-2-yl)- | D |
| | N-p-tolyl-acetamide | |
| 40 | N-Benzyl-2-(1-methyl-3-phenylsulfanyl-1H- | D |
| | indol-2-yl)-acetamide | |
| 41 | N-(4-Chloro-phenyl)-2-(1-methyl-3- | D |
| | phenylsulfanyl-1H-indol-2-yl)-acetamide | |
| 42 | N-(3-Hydroxy-propyl)-2-(1-methyl-3- | D |
| | phenylsulfanyl-1H-indol-2-yl)-acetamide | |
| 43 | 2-(1-Benzyl-3-phenylsulfanyl-1H-indol-2-yl)- | D |
| | N-(3-hydroxy-propyl)-acetamide | |
| 44 | 2-(1-Benzyl-3-methylsulfanyl-1H-indol-2-yl)- | D |
| | N-(4-methoxy-phenyl)-acetamide | |
| 45 | 2-(1-Benzyl-1H-indol-2-yl)-N-(4-methoxy- | D |
| | phenyl)-acetamide | |
| 46 | 2-(1-Methyl-3-methylsulfanyl-1H-indol-2-yl)- | D |
| | N-p-tolyl-acetamide | |
| 47 | 2-(1-Benzyl-3-methylsulfanyl-1H-indol-2-yl)- | D |
| | N-(2-chloro-phenyl)-acetamide | |
| 48 | 2-(1,5-Dimethyl-3-methylsulfanyl-1H-indol-2- | C |
| | yl)-N-(2-hydroxy-ethyl)-acetamide | |
| 49 | 2-(1-Benzyl-1H-indol-2-yl)-N-(2-chloro- | D |
| | phenyl)-acetamide | |

*Compounds having activity designated with an A have an IC₅₀ of less than 1.0 μM. Compounds having activity designated with a B have an IC₅₀ between 1.0 μM and 10.0 μM. Compounds having activity designated with a C have an IC₅₀ greater than 10.0 μM. Compounds designated with a D were not tested in this assay.

The synthesis and calculations of the IC₅₀ for these compounds are described in U.S. patent application Ser. No. 11/018,018 (filed on Dec. 20, 2004).

Additional exemplary compounds that can be used as a SIRT1 inhibitor are described in WO 03/046207. Some exemplary compounds have the structure of Formula

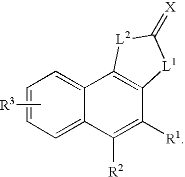

### V:

In Formula V, the letter X is a member selected from the group consisting of O and S. The symbols L¹ and L² each represent members independently selected from the group consisting of O, S, ethylene and propylene, substituted with 0-2 R groups, wherein exactly one of the symbols L¹ and L² represents a member selected from the group consisting of O and S. Each instance of the letter R of symbols L¹ and L² independently represents a member selected from the group consisting of C₁₋₆alkyl, C₂₋₆alkenyl and —CO₂R⁴. The symbols R¹ and R² each represent members independently selected from the group consisting of hydrogen, C₁₋₆alkoxy, C₀₋₆alkoxy-aryl and hydroxy. Alternatively, the symbols R¹ and R² are taken together with the carbons to which they are attached to form a six-membered lactone ring.

The symbol R³ represents a member selected from the group consisting of hydrogen, C₁₋₆alkyl, aryl, —OR⁴, —NR⁴R⁴, —CO₂R⁴, —C(O)R⁴, —C(O)NR⁴R⁴, —CN, —NO₂ and halogen. Each instance of the symbol R⁴ independently represents a member selected from the group consisting of hydrogen and C₁₋₆alkyl.

The compound of Formula V can have the following structure:

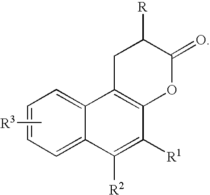

In this case, the symbol R¹ is a member selected from the group consisting of hydrogen, C₁₋₆alkoxy and C₀₋₆alkoxy-aryl; the symbol R² is a member selected from the group consisting of hydrogen and hydroxy; the symbol R³ is a member selected from the group consisting of hydrogen and —OR⁴; and the symbol R⁴ is C₁₋₆alkyl.

In another variation, the symbol R¹ is a member selected from the group consisting of C₁₋₆alkoxy, C₀₋₆alkoxy-aryl and hydroxy. For example, the symbol R¹ is a member selected from the group consisting of hydroxy, methoxy and benzyloxy. In another preferred embodiment, the term aryl is a member selected from the group consisting of phenyl and naphthyl.

Another exemplary compound has the structure of Formula VI:

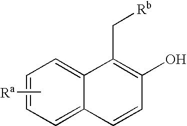

In Formula VI, the symbol R^{a} is a member selected from the group consisting of hydrogen, C₁₋₆alkyl, aryl, —OR^{e}, —NR^{e}R^{e}, —CO₂R^{e}, —C(O)R^{e}, —C(O)NR^{e}R^{e}, —CN, —NO₂ and halogen, while the symbol R^{b} is a member selected from the group consisting of:

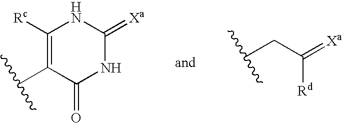

In the components above, the symbol X^{a} can be O, S, or NR^{e}. The symbol R^{c} can be hydrogen, C₁₋₆alkyl and aryl optionally substituted with a hydrogen, C₁₋₆alkyl, aryl, -Ore, —NR^{e}R^{e}, —CN, —NO₂ or halogen. The symbol R^{d} can be hydrogen, C₁₋₆alkyl, aryl, -Ore, —NR^{e}R^{e}, or halogen. Each instance of the symbol R^{e} can be independently hydrogen or C₁₋₆alkyl. In one embodiment, a compound of Formula VI has the following structure

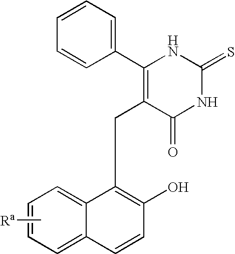

These compounds are described further in WO 03/046207 and U.S. patent application Ser. No. 10/885,997 (filed on Jul. 6, 2004; published as U.S. 2005-0136429 on Jun. 23, 2005).

Still other exemplary SIRT1 inhibitors include Compound A3 (8,9-dihydroxy-6H-(1)benzofuro[3,2-c]chromen-6-one), Compounds M15 (1-[(4-methoxy-2-nitro-phenylimino)-methyl]-naphthalene-2-ol) and Sirtinol (2-[(2-hydroxy-naphthalen-1-ylmethylene)-amino]-N-(1-phenyl-ethyl)-benzamide). Such compounds are available, e.g., from ChemBridge or can be synthesized. See, e.g., Grozinger et al. J. Biol. Chem., 276:38837-3884, 2001. Still other SIRT1 inhibitors include genes that produce anti-sense nucleic acids that inhibit SIRT1 gene expression and other inhibitor agents that can inhibit SIRT1 gene expression, e.g., an inhibitor nucleic acid such as an siRNA, anti-sense RNA, or PNA. Such nucleic acids can be designed to be complementary to a region of the SIRT1 mRNA, e.g., near the initiator methionine codon to inhibit translation or expression of the mRNA.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Techniques useful for the separation of isomers, e.g., stereoisomers are within skill of the art and are described in Eliel, E. L.; Wilen, S. H.; Mander, L. N. Stereochemistry of Organic Compounds, Wiley Interscience, NY, 1994. For example, compound 3 or 4 can be resolved to a high enantiomeric excess (e.g., 60%, 70%, 80%, 85%, 90%, 95%, 99% or greater) via formation of diasteromeric salts, e.g. with a chiral base, e.g., (+) or (−) α-methylbenzylamine, or via high performance liquid chromatography using a chiral column. In some embodiments, the crude product 4, is purified directly on a chiral column to provide enantiomerically enriched compound.

For purposes of illustration, enantiomers of compound 4 are shown below.

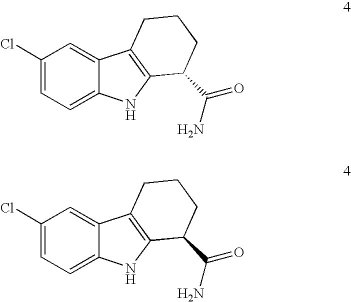

In some instances, the compounds disclosed herein are administered where one isomer (e.g., the R isomer or S isomer) is present in high enantiomeric excess. In general, the isomer of compound 4 having a negative optical rotation, e.g., −14.1 (c=0.33, DCM) or [α]_{D}²⁵-41.18° (c 0.960, CH₃OH) has greater activity against the SirT1 enzyme than the enantiomer that has a positive optical rotation of +32.8 (c=0.38, DCM) or [α]_{D}²⁵+22.720 (c 0.910, CH₃OH). Accordingly, in some instances, it is beneficial to administer to a subject a compound 4 having a high enantiomeric excess of the isomer having a negative optical rotation to treat a disease.

While the enantiomers of compound 4 provide one example of a stereoisomer, other stereoisomers are also envisioned, for example as depicted in compounds 6 and 7 below.

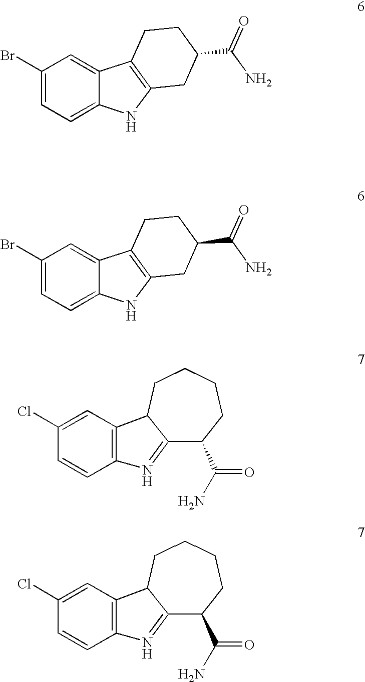

As with the compound of formula 4, in some instances it is beneficial to administer to a subject an isomer of compounds 6 or 7 that has a greater affinity for SirT1 than its enantiomer (i.e., an enantiomerically enriched preparation). For example, in some instances, it is beneficial to administer a compound 7, enriched with the (−) optical rotamer, wherein the amide (or other substituent) has the same configuration as the negative isomer of compound 4.

In some instances, it is beneficial to administer a compound having the one of the following structures where the stereochemical structure of the amide (or other substituent) corresponds to the amide in compound 4 having a negative optical rotation (i.e., an enantiomeric enriched compound).

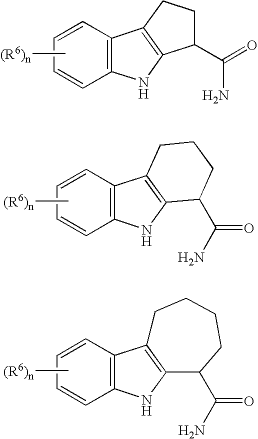

(n is an integer from 0 to 4.)

Salts of the SIRT1 inhibitors (e.g., pharmaceutically-acceptable salts) include those derived from inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N-(alkyl)4+ salts. It is also possible to have the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The amount of active ingredient that may be combined with the carrier materials to produce a single dosage form will vary depending upon the cell being cultured and other culture conditions. A typical preparation will contain from about 5% to about 95% active compound (w/w). Alternatively, such preparations contain from about 20% to about 80% active compound. Lower or higher doses than those recited above may be required.

### In Vitro Assays

The ability of a compound to inhibit SIRT1 can be evaluated in many ways. In some embodiments, interaction with, e.g., binding of, SIRT1 can be assayed in vitro. The reaction mixture can include a SIRT1 co-factor such as NAD and/or a NAD analog. In other embodiments, the ability of the compound to inhibit an enzymatic function, e.g., deacetylase activity of SIRT1, can be assayed in vitro. Assays can include determining the IC₅₀ of the compound.

Examples of enzymatic assays and additional assays are described in U.S. patent application Ser. No. 10/940,269 (filed on Sep. 13, 2004), U.S. patent application Ser. No. 11/018,018 (filed on Dec. 20, 2004), WO 03/046207, and U.S. patent application Ser. No. 10/885,997 (filed on Jul. 6, 2004; published as U.S. 2005-0136429 on Jun. 23, 2005).

An exemplary assay method includes a multi-well format of the SirT1 enzymatic assay that is based on the commercial “Fluor-de-Lys” assay principle by Biomol, which is fluorogenic (www.biomol.com/store/Product_Data_PDFs/ak500.pdf). In this assay, deacetylation of the s-amino function of a lysyl residue is coupled to a fluorogenic development step that is dependent on the unblocked ε-amino functionality and generates fluorescent aminomethylcoumarin. Fluorescence can be read on a commercial macroscopic reader. Standard enzymological analyses can be used to determine Kᵢ. In a preferred embodiment, the assay includes contacting the SIRT1 protein or biologically active portion thereof with a known compound which binds a SIRT1 to form an assay mixture, contacting the assay mixture with a test compound, and determining the ability of the test compound to interact with a SIRT1 protein, wherein determining the ability of the test compound to interact with the SIRT1 protein includes determining the ability of the test compound to preferentially bind to the SIRT1 or biologically active portion thereof, or to modulate the activity of a target molecule, as compared to the known compound.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci 18:284-287, 1993); chromatography (gel filtration chromatography, ion-exchange chromatography); electrophoresis (see, e.g., Ausubel, F. et al., eds. Current Protocols in Molecular Biology 1999, J. Wiley: New York); and immunoprecipitation (see, for example, Ausubel, F. et al., eds. Current Protocols in Molecular Biology, 1999, J. Wiley: New York; Heegaard, N. H., J Mol Recognit 11:141-148, 1998; Hage, D. S., and Tweed, S. A., J Chromatogr B Biomed Sci Appl. 699:499-525, 1997). Further, fluorescence energy transfer may also be conveniently utilized, as described herein, to detect binding without further purification of the complex from solution.

SIRT1
An exemplary SIRT1 sequence is:

(SEQ ID NO:1)

MADEAALALQPGGSPSAAGADREAASSPAGEPLRKRPRRDGPGLERSPGE
PGGAAPEREVPAAARGCPGAAAAALWREAEAEAAAAGGEQEAQATAAAGE
GDNGPGLQGPSREPPLADNLYDEDDDDEGEEEEEAAAAAIGYRDNLLFGD
EIITNGFHSCESDEEDRASHASSSDWTPRPRIGPYTFVQQHLMIGTDPRT
ILKDLLPETIPPPELDDMTLWQIVINILSEPPKRKKRKDINTIEDAVKLL
QECKKIIVLTGAGVSVSCGIPDFRSRDGIYARLAVDFPDLPDPQAMFDIE
YFRKDPRPFFKFAKEIYPGQFQPSLCHKFIALSDKEGKLLRNYTQNIDTL
EQVAGIQRIIQCHGSFATASCLICKYKVDCEAVRGDIFNQVVPRCPRCPA
DEPLAIMKPEIVFFGENLPEQFHRAMKYDKDEVDLLIVIGSSLKVRPVAL
IPSSIPHEVPQILINREPLPHLHFDVELLGDCDVIINELCHRLGGEYAKL
CCNPVKLSEITEKPPRTQKELAYLSELPPTPLHVSEDSSSPERTSPPDSS
VIVTLLDQAAKSNDDLDVSESKGCMEEKPQEVQTSRNVESIAEQMENPDL
KNVGSSTGEKNERTSVAGTVRKCWPNRVAKEQISRRLDGNQYLFLPPNRY
IFHGAEVYSDSEDDVLSSSSCGSNSDSGTCQSPSLEEPMEDESEIEEFYN
GLEDEPDVPERAGGAGFGTDGDDQEAINEAISVKQEVTDMNYPSNKS

Sirtuins are further described, and additional exemplary sequences are presented in U.S. patent application Ser. No. 11/018,018 (filed on Dec. 20, 2004). Sirtuins are members of the Silent Information Regulator (SIR) family of genes.

Natural substrates for SIRT1 include, for example, histones and p53. SIRT1 proteins bind to a number of other proteins, referred to as “SIRT1 binding partners.” For example, SIRT1 binds to p53 and plays a role in the p53 pathway, e.g., K₃₇₀, K₃₇₁, K₃₇₂, K₃₈₁, and/or K₃₈₂ of p53 or a peptide that include one or more of these lysines. For example, the peptide can be between 5 and 15 amino acids in length. SIRT1 proteins can also deacetylate histones. For example, SIRT1 can deacetylate lysines 9 or 14 of histone H3 or small peptides that include one or more of these lysines. Histone deacetylation alters local chromatin structure and consequently can regulate the transcription of a gene in that vicinity. Many of the SIRT1 binding partners are transcription factors, e.g., proteins that recognize specific DNA sites. Interaction between SIRT1 and SIRT1 binding partners can deliver SIRT1 to specific regions of a genome and can cause local changes to the acetylation of substrates, e.g., histones and transcription factors localized to the specific region. It has been found that a genetic deficiency of SIRT1 in mouse embryonic fibroblasts dramatically increases resistance to replicative senescence (Chua et al., Cell Metabolism 2:67, 2005).

### Pharmaceutical Compositions

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-o naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g., L-arginine, -lysine, -histidine salts).

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions delineated herein include the compounds of the formulae delineated herein, as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

A number of embodiments of the invention have been described. Nevertheless, it will be understood that various modifications may be made without departing from the spirit and scope of the invention.

What is claimed is:

1. A method of culturing cells in culture medium, the method comprising
inhibiting SIRT1 activity in cells in vitro, wherein the culture medium comprises a SIRT1 inhibitor having Formula (I) 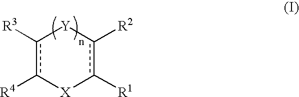
wherein,
R¹ and R², together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which may be optionally substituted with 1-5 R⁵; or R¹ is H, S-alkyl, or S-aryl, and R² is amidoalkyl wherein the nitrogen is substituted with alkyl, aryl, or arylalkyl, each of which is optionally further substituted with alkyl, halo, hydroxy, or alkoxy;
R³ and R⁴, together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which are optionally substituted with 1-5 R⁶;
each of R⁵ and R⁶ is, independently, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C7-C12 heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, SO₃H, sulfate, S(O)NH₂, S(O)₂NH₂, phosphate, C₁-C₄ alkylenedioxy, oxo, acyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl; alkoxyaminocarbonyl; or one of R⁵ or R⁶ and R⁷ form a cyclic moiety containing 4-6 carbons, 1-3 nitrogens, 0-2 oxygens and 0-2 sulfurs, which are optionally substituted with oxo or C₁-C₆ alkyl;
X is NR⁷, O, or S; Y is NR^{7′}, O or S;
———— represent optional double bonds;
each of R⁷ and R^{7′} is, independently, hydrogen, C₁-C₆ alkyl, C₇-C₁₂ arylalkyl, C₇-C₁₂ heteroarylalkyl; or R⁷ and one of R⁵ or R⁶ form a cyclic moiety containing 4-6 carbons, 1-3 nitrogens, 0-2 oxygens and 0-2 sulfurs, which are optionally substituted with oxo or C₁-C₆ alkyl; and
n is 0 or 1.

2. The method of claim 1, wherein, prior to culturing, the cells are senescent or terminally differentiated.

3. The method of claim 1, wherein the SIRT1 inhibitor prolongs lifespan of the cells.

4. The method of claim 1, wherein the cells are bone marrow cells, cardiac muscle cells, dopamine-producing cells, osteoblasts, osteocytes, hepatocytes, stromal cells, fetal brain cells, pancreatic B cells, or myoblasts.

5. The method of claim 1, wherein the cells are stem cells.

6. The method of claim 5, wherein the stem cells are committed to a mesenchymal, hematopoietic, adipogenic, hepatogenic, neurogenic, gliogenic, chondrogenic, vasogenic, myogenic, chondrogenic, or osteogenic lineage.

7. The method of claim 1, wherein the cells are administered to a subject who has experienced or is at risk of experiencing abnormal senescence, diabetes, metabolic syndrome, skeletal muscle disease, ALS under neurodegenerative disease, spinal cord trauma, heart disease, stroke, macular degeneration, a chronically degenerative disease, or other condition characterized by unwanted cell loss, or a subject who has undergone chemotherapy or radiation treatment, a subject that has suffered a wound, a burn, an ulcer, a sore, or abrasions.

8. The method of claim 1, further comprising the step of evaluating one or more test compounds by contacting the test compound to the cells.

9. The method of claim 1, wherein R¹ and R², together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which may be optionally substituted with 1-5 R⁵.

10. The method of claim 1, wherein R¹ and R², together with the carbons to which they are attached, form C₅-C₁₀ cycloalkenyl.

11. The method of claim 10, wherein R¹ and R² are substituted with R⁵.

12. The method of claim 11, wherein R⁵ is an aminocarbonyl.

13. The method of claim 11, wherein R⁵ is an amino substituent.

14. The method of claim 1, wherein R³ and R⁴, together with the carbons to which they are attached, form C₆-C₁₀ aryl.

15. The method of claim 14, wherein R³ and R⁴ are substituted with R⁶.

16. The method of claim 15, wherein R⁶ is halo or C₁-C₆ alkyl.

17. The method of claim 1, wherein n is 0.

18. The method of claim 1, wherein X is NR⁷.

19. The method of claim 1, wherein n is 0 and X is NR⁷.

20. The method of claim 1, the compound having the formula (X) below:

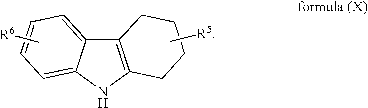

21. The method of claim 20, wherein R⁶ is halo or C₁-C₆ alkyl.

22. The method of claim 20, wherein R⁵ is aminocarbonyl.

23. The method of claim 20, the compound having the formula (XI) below:

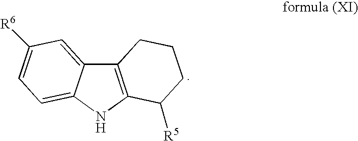

24. The method of claim 23, wherein R⁶ is halo or alkyl.

25. The method of claim 23, wherein R⁵ is aminocarbonyl.

26. The method of claim 23, wherein R⁶ is halo or alkyl and wherein R⁵ is aminocarbonyl.

27. The method of claim 20, wherein the compound is 6-Chloro-2,3,4,9-tetrahydro-1H-carbazole-1-carboxylic acid amide.

28. The method of claim 27, wherein the compound comprises greater than a 60% enantiomeric excess of the enantiomer having an optical rotation of −14.1 (c=0.33 DCM).

29. The method of claim 28, wherein the compound comprises greater than a 90% enantiomeric excess of the enantiomer having an optical rotation of −14.1 (c=0.33 DCM).
